(19) United States
(12) Patent Application Publication
(10) Pub. No.: US 2008/0318208 A1
(43) Pub. Date: Dec. 25, 2008

(54) Dengue Reporter Virus and Methods of Making and Using the Same

(75) Inventors: Bridget Puffer, Devon, PA (US); Benjamin J. Doranz, Drexel Hill, PA (US)

Correspondence Address: Pepper Hamilton, LLP, 400 Berwyn Park, 899 Cassatt Road, Berwyn, PA, 19312-1183, US

(73) Assignee: Integral Molecular, Inc., Philadelphia, PA (US)

(21) Appl. No.: 12/279,134

(22) PCT Filed: Feb. 12, 2007
(86) PCT No.: PCT/US07/03660

§ 371 Date: Aug. 28, 2008

Related U.S. Application Data
(60) Provisional application No. 60/772,916, filed on Feb. 13, 2006.

(51) Int. Cl. C12Q 1/70; C12P 21/04; C12N 15/00; C12N 5/06; C12N 15/87

(52) U.S. Cl. 435/5; 435/320.1; 435/69.1; 435/325; 435/456

(57) ABSTRACT

The present invention relates to the production and uses of Dengue virus replicons and Dengue reporter virus particles. The present invention relates to methods of identifying inhibitors of Dengue virus infection, inhibitors of Dengue virus replication, and inhibitors of Dengue virus assembly.

### CROSS REFERENCE TO RELATED APPLICATIONS

This application claims priority to U.S. Provisional Application Ser. No. 60/772,916 filed Feb. 13, 2006, which is herein incorporated by reference in its entirety.

### GOVERNMENT SUPPORT

This invention was made with U.S. Government support (NIH Grant No. A1062100) and the U.S. Government may therefore have certain rights in the invention.

### BACKGROUND OF THE INVENTION

Flaviviruses have a global impact due to their widespread distribution and ability to cause encephalitis in humans and economically important domesticated animals. Of the approximately seventy viruses in the genus, roughly half have been associated with human disease. Several members of this group, such as dengue virus (DEN) and West Nile virus (WNV), are considered emerging or re-emerging pathogens because the incidence with which they encounter humans and cause disease is increasing each year at an alarming rate. Globally, DEN has become the most significant source of arthropod-borne viral disease in humans. Approximately 2.5 billion people (40% of the world's population) live at risk for DEN exposure across the globe, resulting in more than 100 million cases of DEN related illnesses each year.

The genome of flaviviruses such as DEN is a positive-stranded RNA. In the presence of non-structural proteins encoded by the virus, the RNA can be replicated within the cytoplasm of a host cell. A nucleic acid molecule that codes for all the proteins necessary for its replication in a cell is termed a “replicon”. If RNA encoding the DEN replicon is transfected into cells, the replicon can replicate. RNA-based replicons of Kunjin virus that carry a reporter gene have been described (Khromykh, et al. (1998), J Virol, 72:5967-77, Khromykh, et al. (1997), J Virol, 71:1497-505, Varnavski, et al. (1999), Virology, 255:366-75, Westaway, et al. (2005)) (U.S. Pat. No. 6,893,866). Such replicons can be transfected into stable or inducible cell lines to produce reporter viruses (Harvey, et al. (2004), J Virol, 78:531-8). Subgenomic replicons of Dengue virus have also been described (Holden, et al. (2006), Virology, 344:439-52) (Pang, et al. (2003)) (U.S. Patent Application No. 2004/0265338). A plasmid carrying a DNA-based version of a replicon that could be transfected into a cell directly (rather than an RNA transcript from the DNA) has been described for West Nile virus (Pierson, et al. (2005), Virology). Replication-competent clones of West Nile virus have also been described that carry a green fluorescent protein (GFP) reporter virus (Pierson, et al. (2005), Virology, 334:28-40).

Four different serotypes of DEN are transmitted to humans through the bite of Aedes aegypti and Aedes albopictus mosquitoes. Clinical manifestations of exposure to DEN vary significantly (for review see (Gibbons, et al. (2002), Bmj, 324:1563-6)). Common clinical manifestations of dengue fever (DF) include a febrile illness accompanied by retroorbital, muscle and joint pain. While primary exposure to DEN is not associated with significant mortality, a small percentage of exposed individuals experience a more severe disease course referred to as dengue hemorrhagic fever (DHF). DHF, which is fatal in up to 10% of affected individuals, is most common in individuals that are sequentially infected with multiple different serotypes of the virus. Of significant concern is the rapid increase in the number of DHF cases during the past twenty years, resulting in over 450,000 cases of DHF each year (Monath, et al. (1996), Fields Virology, 2:961-1034). The increasingly common spread of different dengue serotypes is expected to increase the frequency of DHF significantly.

Dengue viruses are small spherical virions composed of three viral structural proteins, a lipid envelope, and a copy of the RNA genome (Kuhn, et al. (2002), Cell, 108:717-25, Mukhopadhyay, et al. (2003), Science, 302:248, Zhang, et al. (2003), Embo J, 22:2604-13). The cell biology of DEN entry into cells is poorly understood. To date, a cellular receptor for DEN has not yet been identified, although recent evidence suggests a role for DC-SIGN and/or DC-SIGNR during attachment and entry into primary dendritic cells (Navarro-Sanchez, et al. (2003), EMBO Rep, 4:723-8, Tassaneetrithep, et al. (2003), J Exp Med, 197:823-9). The role of the receptor is to bind virus particles on the cell surface and deliver them into the mildly acidic endosomal compartments of the cell, where the envelope proteins of the virus mediate fusion in a pH-dependent fashion.

The positive sense RNA genome of DEN is approximately 11 kb in length and encodes a single polyprotein that is cleaved by cellular and viral proteases into ten smaller functional subunits: three structural and seven non-structural (NS) proteins (Khromykh, et al. (1999), J Virol, 73:10272-80, Khromykh, et al. (2000), J Virol, 74:3253-63, Rice (1996), Fields Virology, 2:931-959). The structural proteins of DEN, which include the capsid, pre-membrane (prM) and envelope (E) proteins, are synthesized at the amino-terminus of the polyprotein and are present in the mature virus particle. The seven non-structural proteins encode all the enzymatic functions required for replication of the DEN genomic RNA, including a RNA-dependent RNA polymerase (NS5) (Rice (1996), Fields Virology, 2:931-959). The sequence encoding the DEN polyprotein is flanked by two untranslated regions (UTRs) that are required for efficient translation and genomic RNA replication (Khromykh, et al. (2003), J Virol, 77:10623-9, Khromykh, et al. (2000), J Virol, 74:3253-63, Novak, et al. (1994), Genes Dev, 8:1726-37). DEN RNA replication occurs in the cytoplasm at specialized virus-induced membrane structures (Mackenzie, et al. (1999), J Virol, 73:9555-67, Mackenzie, et al. (1998), Virology, 245:203-15). Viral particle biogenesis and budding occurs at the endoplasmic reticulum, and viruses are released through the secretory pathway of the cell (Lorenz, et al. (2003), J Virol, 77:4370-82, Mackenzie, et al. (2001), J Virol, 75:10787-99).

The ability of enveloped viruses to enter permissive cells is conferred by envelope glycoproteins incorporated into the viral membrane. Class II envelope proteins, encoded by the alpha- and flaviviruses, describe those that contain an internal fusion loop, lie flat across the surface of the native virion as dimers, and do not appear to form coiled-coils while mediating lipid mixing and fusion (reviewed in (Heinz, et al. (2000), Adv Virus Res, 55:231-69)). Like other class II fusion systems, DEN entry and fusion involves two separate proteins. The E protein plays a central role in virus entry by virtue of its capacity to bind receptor and mediate fusion in a pH-dependent fashion. The primary role of the second protein, prM, involves protecting newly formed particles from irreversible premature inactivation as they transit through mildly acidic compartments in the secretory pathway (Zhang, et al. (2003), Embo J, 22:2604-13). Other functions of prM have been demonstrated including directing E protein folding and trafficking (Lorenz, et al. (2002), J Virol; 76:5480-91). Structural studies suggest that all class II fusion proteins share a common structural design.

DEN virions are small spherical particles (50 nM) comprised of a lipid envelope incorporating 180 E glycoproteins arranged in a herringbone configuration (Kuhn, et al. (2002), Cell, 108:717-25). The capsid, prM and E components assemble at the endoplasmic reticulum to form an immature particle that buds into the lumen of the ER. Cleavage of the prM protein by the furin protease during trafficking to the cell surface (to generate the M protein), activates the fusion potential of the E protein, allowing the conformational changes that mediate fusion to occur upon exposure to low pH (Elshuber, et al. (2003), J Gen Virol, 84:183-91). Interestingly, expression of prM-E alone is sufficient for the production and secretion of subviral particles (SVPs) that, despite being smaller than mature viruses, retain the ability to mediate fusion in a manner analogous to mature particles containing capsid (Corver, et al. (2000), Virology, 269:37-46, Ferlenghi, et al. (2001), Mol Cell, 7:593-602, Heinz, et al. (1995), Vaccine, 13:1636-42). The ability to form subviral particles in the absence of any other viral proteins suggests that the forces that drive the process of particle biogenesis and budding reside in prM-E. Mature Dengue virus particles are approximately 50 nM in diameter and contain multiple copies of the viral capsid and the viral genomic RNA. Smaller 30 nM particles composed of prM-E proteins, called subviral particles, are also produced during virus infection. While subviral particles do not contain RNA or capsid, the E proteins on these particles are able to mediate receptor binding and fusion.

A primary target for neutralizing antibodies in a flavivirus infected host is the E glycoprotein present on the surface of the virus particle (Monath, et al. (1996), Fields Virology, 2:961-1034). Additionally, antibodies generated against prM and nonstructural protein-1 (NS1) have also been observed. Several lines of evidence support a significant role for such antibodies during virus clearance and the establishment of immunity following vaccination. For example, passive transfer of antibodies has been shown to confer protection in experimental systems with several flaviviruses, including tick bourne encephalitis (TBE), yellow fever virus (YF), Japanese encephalitis virus (JEV), WNV, and Saint Louis encephalitis virus (SLE). Studies in murine and hamster systems of WNV infection have reached similar conclusions. Several vaccine approaches are being developed, including the use of inactivated virus particles, live attenuated viruses, non-infectious subviral particles, subunit, and nucleic acid vaccines (Pugachev, et al. (2003), Int J Parasitol, 33:567-82). In many of these studies, particularly those in humans, the development of neutralizing antibodies is employed as a correlate of immunity and a measure of efficacy.

The development of a vaccine for DEN has been a significant challenge and the focus of considerable effort (Monath, et al. (1996), Fields Virology, 2:961-1034). While antibodies play a significant role in DEN immunity, the presence of DEN antibodies has also been linked to a more severe clinical outcome due to the ability of antibodies to facilitate DEN infection under some circumstances. While natural infection with one serotype of DEN results in generation of humoral immunity that protects against subsequent challenge with a homotypic virus, protection against other serotypes is transient. In fact, sequential exposure to different serotypes of DEN increases the likelihood of developing DHF. Pioneering work by Halstead and colleagues suggest that the presence of antibodies raised against the first serotype of DEN significantly impacts the outcome of a second exposure by allowing antibody dependent enhancement (ADE) of infection and the activation of both complement and the cellular immune system (Halstead (1988), Science, 239:476-81, Halstead (1989), Rev Infect Dis, 11 Suppl 4:S830-9, Halstead, et al. (1970), Yale J Biol Med, 42:311-28, Halstead, et al. (1977), J Exp Med, 146:201-17, Kliks, et al. (1989), Am J Trop Med Hyg, 40:444-51, Mongkolsapaya, et al. (2003), Nat Med, 9:921-7). Together, ADE has been linked to an increase in viral burden, increased vascular permeability, and a more severe disease course. One implication of these studies is that great care must be taken in the design of a vaccine against DEN to avoid a strategy that confers protection to only one serotype. Protection against only a single DEN serotype would increase the likelihood of an individual's chance of developing DHF should they encounter a second serotype of DEN. A tetravalent vaccine that simultaneously protects against all four serotypes of DEN is needed. Thus, characterizing not only the magnitude, but also the breadth, persistence, and specificity of the humoral response in response to vaccination is an important component of evaluating candidate vaccines and understanding pathogenesis in naturally infected individuals.

The standard method for detecting neutralizing antibodies to DEN is the plaque reduction neutralization test (PRNT) (Monath, et al. (1996), Fields Virology, 2:961-1034, Russell, et al. (1967), J Immunol, 99:291-6). Using this approach, the ability of an antibody to bind virus and neutralize its infectivity is measured as a reduction in the number of plaques formed following infection and subsequent propagation in cell culture. The PRNT approach involves the use of live infectious virus, and requires about a week for plaque formation and analysis. The quantitative power of plaque assays is limited by the number of wells examined and the number of plaques counted by the investigator. The latter process is somewhat subjective when plaque size and morphology is variable. The ability of flaviviruses to form plaques in infected cell monolayers is cell type-, and virus strain-dependent. Thus, the PRNT approach does not allow for the neutralizing capacity of antibodies to be detected using strains that plaque poorly, or on all permissive cell types, excluding many that may be relevant in vivo.

There is a need for better methods and compositions for the generation of pharmaceuticals and vaccines against flaviviruses, such as Dengue. The present invention fulfills these needs as well as others.

### SUMMARY OF THE INVENTION

In some embodiments, the present invention provides isolated nucleic acid molecules encoding a replicon of DEN under the control of a eukaryotic promoter.

In some further embodiments, the present invention provides isolated nucleic acid molecules encoding a replicon of DEN wherein said DNA molecule comprises nucleic acid encoding a reporter.

In some further embodiments, the present invention provides isolated nucleic acid molecules encoding a replicon of DEN wherein said reporter is selected from the group consisting of a GFP reporter, a Renilla luciferase reporter, and a beta-galactosidase reporter.

In some further embodiments, the present invention provides isolated nucleic acid molecules encoding a replicon of DEN wherein said DNA molecule is free of nucleic acid encoding at least one full-length structural protein of DEN.

In some further embodiments, the present invention provides isolated nucleic acid molecules encoding a replicon of DEN wherein said DNA molecule comprises nucleic acid encoding at least a portion of one structural protein of DEN selected from the group consisting of C, prM, E.

In some embodiments, the present invention provides methods of producing DEN reporter virus particles (RVPs) comprising the step of contacting a cell in reporter virus particle media with a DNA molecule encoding a replicon of DEN and a reporter, wherein said cell takes up the DNA molecule, expresses said replicon of DEN and said reporter, and produces DEN RVPs.

In some further embodiments, the present invention provides methods of producing DEN RVPs wherein said DNA molecule comprising a replicon of DEN is a plasmid.

In some further embodiments, the present invention provides methods of producing DEN RVPs wherein the reporter virus particle media is maintained at a pH of about 7.5 to about 8.5.

In some further embodiments, the present invention provides methods of producing DEN RVPs wherein the reporter virus particle media is maintained at a pH of about 7.5 to about 8.5.

In some further embodiments, the present invention provides methods of producing DEN RVPs wherein the reporter virus particle media is maintained at pH of about 8.

In some further embodiments, the present invention provides methods of producing DEN RVPs wherein said contacting comprises transfection of said plasmid.

In some further embodiments, the present invention provides methods of producing DEN RVPs wherein DNA molecule is free of nucleic acid sequences encoding at least one full-length structural protein of DEN.

In some further embodiments, the present invention provides methods of producing DEN RVPs wherein said cell stably expresses or inducibly expresses the C, prM, and E proteins of DEN.

In some further embodiments, the present invention provides methods of producing DEN RVPs wherein the DEN RVPs are harvested between 72 hours and 148 hours after contact between said DNA molecule and said cell In some embodiments, the present invention provides cells comprising structural proteins of Dengue and is free of the non-structural proteins of Dengue.

In some embodiments, the present invention provides methods of producing DEN RVPs comprising the steps of: a) contacting a cell in reporter virus particle media with the DNA molecule of claim 1 wherein said cell comprises (i) nucleic acids that encode DEN structural proteins; and (ii) an inducible promoter that controls the expression of DEN structural proteins; and b) inducing expression of DEN structural proteins in said cells, wherein said inducing expression of DEN structural proteins produces said RVPs.

In some further embodiments, the present invention provides methods of producing DEN RVPs wherein reporter virus particle media that is maintained at pH of about 7.5 to about 8.5 during RVP production.

In some embodiments, the present invention provides methods of producing DEN RVPs wherein reporter virus particle media is maintained at pH of about 8.

In some embodiments, the present invention provides methods of producing DEN RVPs wherein the DEN RVPs are harvested between 72 hours and 148 hours after contact between said DNA molecule and said cell.

In some embodiments, the present invention provides compositions comprising Dengue reporter virus particles and a storage buffer, wherein said storage buffer is maintained at a pH of about 7.5 to about 8.5.

In some embodiments, the storage buffer further comprises an additive. In some further embodiment, the storage buffer may comprise a protein additive. In some embodiments, the total protein additive concentration of the storage buffer is 8 μg per ml upon addition of a protein additive

In some embodiments, the present invention provides methods of infecting a cell comprising contacting said cell with a Dengue reporter virus particle. In some further embodiments, said cell expresses DC-SIGNR. In some further embodiments, said cell is a Raji-DC-SIGNR.

In some embodiments, the present invention provides methods of identifying a compound that inhibits Dengue infection comprising a) contacting a cell with a Dengue RVP in the presence or absence of a test compound; and b) determining if said Dengue RVP can infect said cell in the presence and absence of said test compound, wherein if the presence of said test compound inhibits the Dengue RVP infection of said cell, said test compound is said to be a compound that inhibits Dengue infection.

In some embodiments, the present invention provides methods of identifying a compound that inhibits Dengue assembly comprising contacting a Dengue RVP producer cell with a test compound and determining if the Dengue RVPs can assemble in the presence of said test compound, wherein if assembly is prevented said test compound is said to be a compound that inhibits Dengue assembly.

In some embodiments, the present invention provides methods of identifying a compound that inhibits DEN RNA replication comprising contacting a cell containing a DEN replicon with a test compound and measuring replicon replication, wherein a decrease in replicon replication indicates that said test compound is a compound that inhibits DEN RNA replication.

In some further embodiments, replicon replication is measured by the expression of a reporter gene. In some further embodiments, said reporter gene is GFP, luciferase, or beta-galactosidase.

In some embodiments, the present invention provides methods of identifying neutralizing antibodies against Dengue virus comprising a) contacting a Dengue RVP with a test antibody; b) contacting the mixture of a) with a cell; and c) measuring the infection of said cell in the presence of said test antibody as compared to the absence of said test antibody, wherein a decrease in infection in the presence of said test antibody indicates that said test antibody is a neutralizing antibody against Dengue virus.

In some embodiments, the DEN RVP comprises a nucleic acid sequence that encodes GFP, luciferase, or beta-galactosidase.

In some embodiments, the composition comprising the test antibody further comprises patient serum.

In some embodiments, the composition comprising the test antibody comprises test antibody is a serotype-specific DEN antibody and said DEN RVP is a serotype-specific DEN RVP.

### BRIEF DESCRIPTION OF THE DRAWINGS

FIG. 1. Schematic diagram of the pDrep2AH-GFP plasmid. Indicated are the CMV promoter, hepatitis delta virus ribozyme, MluI restriction sites, and coding sequences for the twenty-five amino terminal residues of capsid (C25), GFP reporter (E-GFP), foot and mouth disease 2A autoprotease (2a), and non-structural components of the DEN-2 polyprotein. Numbers indicate base pair locations within the plasmid. The plasmid also carries ampicillin resistance (β-lactamase). An expanded schematic depicting the locations of the C25, e-GFP, and 2A protease sequences is shown above the linear diagram. Indicated below the linear sequence indicator is the polyprotein expressed upon translation, which results in a fusion protein comprised of C25, E-GFP, FMDV 2A protease, and the DEN nonstructural proteins.

FIG. 2 (Panels A and B). GFP expression upon transfection of HEK-293T cells with plasmid encoding GFP or replicon. A. HEK-293T cells were plated in 6 well plates at a density of one million cells per well. Cells were transfected with the indicated plasmids using a standard lipofectamine 2000 transfection protocol. Plasmid “cmin HF” encodes a GFP protein under control of a minimal promoter. Plasmids pDR2AH GFP and pDR2AH GFP-Zeo carry dengue replicons that contain the GFP sequence. Cells were imaged using a Nikon Eclipse TE2000U microscope with a Coolpix camera. B. 293T cells were plated in 24 well plates at a density of 0.25×10⁶ cells per well. Cells were transfected with the designated plasmids using a standard calcium phosphate protocol. At the indicated time points, cells were harvested, fixed with 2% paraformaldehyde, and analyzed by flow cytometry for GFP expression to obtain the mean fluorescence intensity.

FIG. 3. Expression of CME proteins in inducible stable cell lines. Stable TREx-293 cells carrying the WestPac, 16681, New Guinea C (NGC), or S16803 CprME coding sequence under regulation of a tetracycline responsive promoter were plated in 6 well plates at a density of 0.5×10⁶ cells per well in the presence or absence of doxycycline (1 μg/ml). 48 hours after plating, the cells were washed with PBS and lysed in PBS, 0.5% TritonX-100. Insoluble material was removed by centrifugation, and 40 μg total protein was analyzed by western blotting with antibodies 4G2 and 2H2. Bands were detected with a horseradish peroxidase (HRP)-conjugated anti-mouse secondary, Supersignal West Pico luminescence reagent, and imaged with an Alphalnnotech Fluorchem 8900. Numbers indicate the samples loaded on the gel as follows: 1. Precision plus molecular weight standards, 2. WestPac induced lysate, 3. Westpac uninduced lysate, 4. 16681 uninduced lysate, 5. 16681 induced lysate, 6. NGC induced lysate, 7. NGC uninduced lysate, 8. S16803 induced lysate, 9. S16803 uninduced lysate, 10. 293TREx parental cell line lysate.

FIG. 4. Infection of Raji DC-SIGN R cells with RVPs harvested at various time points. Stable cell lines carrying the WestPac, S16803 (PDK50), 16681, or New Guinea C (NGC) structural genes (CME) were transfected with pDRep2AH-GFP plasmid. At 4-16 hours post-transfection, the cells were supplemented with doxycycline (1 μg/ml), and subsequently media was harvested at the indicated intervals. RVPs were filtered through a 0.45 μm filter unit and 100 μl aliquots were placed in a 96-well plate. An equal volume of Raji DC-SIGNR cells were then added to the RVPs, at a density of 0.3×10⁶ cells per ml. 48 hours after infection, the cells were fixed with 2% paraformaldehyde and analyzed by flow cytometry for percentage of GFP-positive cells.

FIG. 5. Effect of freezing on RVP infectivity. S16803 RVPs were produced by a standardized protocol and supplemented with equal amounts of additive to the designated concentrations. After 24 hours storage at −80° C., aliquots of each were diluted in series with equal volumes of RPMI-10% FCS, 1% Penicillin-Streptomycin solution, 2 mM L-Alanyl, L-Glutamine dipeptide solution, and 25 mM HEPES pH 8.0. An equal volume of Raji DC-SIGNR cells were then added to the RVPs, at a density of 0.3×10⁶ cells per ml. 48 hours after infection, the cells were fixed with 2% paraformaldehyde and analyzed by flow cytometry. Results were compared to results from the same RVPs tested pre-freezing (not shown).

FIG. 6. Infection of various cell types by RVPs. RVPs carrying the structural proteins of New Guinea C (NGC), 16681, or S16803 were produced. Infection of Raji DC-SIGNR cells was performed as described herein. BHK and Vero cells were cultured in 24-well plates at densities of 30,000 and 20,000 cells per well, respectively. After adherence to plastic, the media was replaced with 200 μl media (DMEM-5% FCS, 1% Penicillin-Streptomycin solution, 2 mM L-Alanyl, L-Glutamine dipeptide solution, and 25 mM HEPES pH 8). An equal volume of the designated RVP was added. Approximately 48 hours post-infection, cells were trypsinized, fixed with 2% paraformaldehyde, and analyzed by flow cytometry for percentage of GFP-positive cells. Results are normalized for the maximum percent infection (100%) in each cell type.

FIG. 7. Neutralization of RVPs by monoclonal antibodies. Neutralization assays were performed by incubation of monoclonal antibodies at the designated concentrations with RVPs undiluted (neat), 2, 4, or 8 fold diluted in complete RPMI (pH=8). RVP and antibody were incubated for one hour at room temperature with shaking, then supplemented with 10 μl Raji DC-SIGNR cells in complete RPMI at a density of 3 million cells per ml (30,000 cells total per well). Plates were returned to a 37° C. incubator for 48 hours, then fixed with 2% paraformaldehyde and analyzed by flow cytometry. Percent infection was calculated by comparison of infectivity observed in the presence of antibody relative to a control without antibody incubation.

FIG. 8. Quantitation of E protein. Plates were coated with monoclonal antibody 3H5, blocked with 2% blotto in PBS 0.1% Tween, and incubated with serial dilutions of purified soluble DEN2 E protein or subviral particles (SVP). Plates were washed with PBS 0.1% Tween, and incubated with biotinylated 4G2 antibody. Washing was repeated and streptavidin-HRP conjugate added. After a final washing, Supersignal Pico was added and signal detected with a luminometer. RLU indicates relative light units. Protein concentration was derived from the amount of purified soluble E protein added. The soluble E was made using recombinant vaccinia and purified over a heparin affinity column and a His tag affinity column. Total protein was determined by BCA assay, and percent of the total protein that was due to E was determined by Sypro staining of the sample in an SDS-PAGE gel and quantifying the percent of protein that was from the E protein band.

FIG. 9. Infectivity of RVPs produced at pH 8.0, 7.2, or below 7.0. RVPs were harvested from culture media at the indicated pHs and used to infect Raji DC-SIGNR cells as described herein. Approximately 48 hours post-infection cells were fixed with 2% paraformaldehyde and analyzed by flow cytometry.

FIG. 10. Monoclonal antibody-mediated enhancement of K562 cell infection by DEN RVPs. Monoclonal antibodies were diluted in complete RPMI, pH of 8, and incubated with S16803 (DEN2) RVPs. Duplicate samples were then mixed with FcR-positive K562 cells (major graph) or FcR-negative RajiDC-SIGNR cells (inset graph) and incubated at 37° C. for 48-72 hours. Cells were then fixed and analyzed for infection by flow cytometry to determine the percentage of GFP-positive cells. Black bars indicate infection of K562 cells; white bars (inset) indicate infection of RajiDC-SIGNR cells.

FIG. 11. Neutralization of DEN1 and DEN2 RVPs by human sera. DEN RVPs were incubated with the designated dilutions of convalescent sera for one hour. Raji DC-SIGNR cells were then added and incubated for 48 hours. Cells were then fixed with paraformaldehyde and analyzed by flow cytometry. Neutralization was calculated as the percent of GFP positive cells observed relative to no sera control wells. D1=anti-Dengue 1 serum. D2=anti-Dengue 2 serum. D1234=serum raised against Dengue 1, 2, 3, and 4 serotypes. JE=negative control serum against a different flavivirus.

FIG. 12. DEN RVP infectivity enhanced in presence of convalescent sera. Designated human sera were serially diluted and incubated with DEN1 (WestPac) or DEN2 (S16803) RVPs. K562 cells were added and cultured for 48-72 hours. Cells were then fixed with paraformaldehyde and analyzed for infection by flow cytometry to determine the number of GFP-positive cells per well.

FIG. 13. Replicon-mediated Renilla luciferase expression in RVP-infected cells. RVPs were produced by transfection of packaging cell lines with the DEN Rep-Renilla replicon plasmid. Cells were induced with doxycycline and RVPs harvested. Serial dilutions of RVPs harvested at 148 hours post-transfection were used to infect Raji DC-SIGNR cells. At 72 hours post-infection, cells were lysed and examined for luciferase activity using the Renilla luciferase assay kit (Promega). Luciferase activity was quantitated with a Wallac Victor luminometer.

FIG. 14. Production of RVPs by cloned cell lines. S16803 and Westpac packaging cells were cloned by limiting dilution, expanded, and tested for RVP production using the DEN GFP replicon. RVP production by uncloned cells was performed in parallel. At 24 hour intervals, media was harvested, filtered and examined for infectivity of Raji DC-SIGNR cells. At 48 hours post-infection, cells were fixed and examined for percent infection by flow cytometry for quantitation of GFP-positive cells. E2, B4, and C3 represent individual cloned cell lines.

FIG. 15. Betagalactosidase expression in cells transfected with DEN Rep-LacZ. HEK-293T cells were transfected with a control plasmid or a DEN Rep-LacZ plasmid. At 24 hours post-transfection cells were fixed with paraformaldehyde and examined for beta-galactosidase activity by X-gal staining. Blue cells indicated beta-galactosidase activity.

### DETAILED DESCRIPTION

In some embodiments, the present invention provides a nucleic acid sequence encoding a replicon of DEN. In some embodiments, a nucleic acid sequence encoding a replicon of the DEN virus comprises the minimal portion of the DEN virus genome capable of self-replication. In some embodiments, the nucleic acid sequence encoding a replicon comprises only the minimal portion of the DEN virus genome capable of self-replication. In some embodiments, the minimal portion does not include the structural proteins of the DEN virus. In some embodiments, the minimal portion comprises a nucleic acid sequence encoding the non-structural proteins of the DEN virus. The nucleic acid molecule can be either DNA or RNA. In some embodiments, the nucleic acid sequence is free of RNA bases. In some embodiments, the DNA encoding the replicon is a plasmid. The nucleic acid sequence can comprise a promoter operably linked to the nucleic acid sequence encoding the replicon. The promoter can be any promoter, including but not limited to promoters that are functional in eukaryotic cells. In some embodiments, the promoter is specifically functional in a eukaryotic cell. In some embodiments, the promoter is, but not limited to a CMV promoter, SV40, and the like. In some embodiments, the promoter is an inducible promoter.

The nucleic acid sequence encoding replicons and the resulting replicons of the present invention can also comprise reporter constructs such that one can monitor the replication or expression of the genes found in the nucleic acid sequence of the replicon. The reporter can also be used to measure infectivity of any virus or virus-like particle that contains the replicon. Examples of reporters include, but are not limited to, a fluorescent reporter, a luciferase reporter, β-Galactosidase reporter, alkaline phosphatase reporter, chloramphenicol acetyltransferase (CAT), and the like. Examples of fluorescent reporters include, but are not limited to, GFP reporter, YFP reporter, and the like. Examples of luciferase reporters include, but are not limited renilla luciferase reporter and firefly luciferase reporter. In some embodiments the replicon comprises a gene that allows for selection of a cell that comprises the replicon. For example, a cell can be selected for comprising the nucleic acid sequence encoding the replicon by contacting the cell with a drug or chemical that because of the presence of the replicon the cell is resistant to the drug or chemical whereas cells that do not contain the replicon will die. Accordingly, in some embodiments, the nucleic acid sequence encoding the replicon comprises a drug resistant gene that allows a cell to escape the effects of drug or chemical. Examples of markers that can be used include, but are not limited to, zeomycin, and the like. Zeocin (zeomycin) is a member of the bleomycin antibiotic family. One could also use hygromycin, neomycin, blasticidin, puromycin, or mycophenolic acid resistance markers and antibiotics and the like as selection markers.

The present invention also provides methods of producing Dengue reporter virus particles (RVPs). A reporter virus particle is a particle that comprises elements of a virus which are produced from a cell comprising a replicon and comprising any other elements necessary for the generation of the virus or virus-like particle. The RVP also comprises a reporter gene. The presence of the reporter gene can be used to monitor the particle's assembly, replication, infection ability, and the like.

In some embodiments, a method of producing Dengue RVPs comprises contacting a cell with a nucleic acid sequence encoding a replicon of the present invention. In some embodiments, the nucleic acid molecule encoding a replicon comprises a DNA molecule that encodes an RNA sequence. The RVPs are then produced once the cell has taken up the replicon.

The nucleic acid molecule encoding the replicon can be contacted with the cell in any manner that enables the nucleic acid molecule encoding the replicon to enter the cell or to be transfected into the cell. Examples of methods of contacting a nucleic acid molecule encoding the replicon with a cell includes, but are not limited to, calcium phosphate transfection, lipid-mediated transfection, electroporation, infection with a virus coding for the replicon, and the like.

In some embodiments, the cell that is contacted with the nucleic acid encoding a replicon comprises elements that can express the structural elements of the virus (e.g. Dengue virus) such that when the replicon is expressed in the cell in conjunction with the structural elements, a RVP is produced. In some embodiments, the structural elements are stably expressed in the cell. Examples of structural elements that can be present in the producer cell include, but are not limited to, Capsid (C), pre-membrane protein (prM), Envelope protein (E), or combinations thereof.

In some embodiments the structural proteins are under control of an inducible promoter such that the expression is regulated by the presence or absence of a compound or other type of molecule. Any inducible promoter can be used. Examples of inducible promoters include, but are not limited to, tetracycline (TREx, Invitrogen), Rheoswitch (NEB), Ecdyson (Invitrogen, Stratagene), Cumate (Qbiogene), glucocorticoid responsive promoter, and the like.

In some embodiments, a producer cell can be used that has the structural proteins stably transfected under the control of an inducible promoter. For example, a HEK-293 cell can stably express the structural proteins of Dengue virus (e.g. C, prM, and E) under the control of a tetracycline inducible promoter. An example of such a cell line is referred to herein as “CME 293trx,” which expresses the capsid, premembrane protein, and envelope protein of Dengue virus under the control of a tetracycline inducible promoter.

When contacting the cells with the replicon the confluence or density of the cells on the plate, well, or other type of container can be modified to increase or decrease transfection efficiency. In some embodiments, the cells are contacted with the replicon when they are at 40-70% or about 50% to about 60%, or 50 to 60% confluence. Additionally, for example for transfection methods using calcium phosphate, the confluence of the cells is about 70%, whereas for cells that are transfected with a lipid mediated agent (e.g. lipofectamine) the cells can be at a confluence of about 90%.

As used herein, the term “about” refers to an amount that is +10% of the amount being modified. For example “about 10” includes from 9 to 11.

In some embodiments, the cell that is contacted with the nucleic acid molecule encoding the replicon is also contacted with reporter virus particle media. The “reporter virus particle media” is media that facilitates or enhances the production of reporter virus particles by maintaining the pH of media in which RVP-producing cells are growing (e.g. in a tissue culture well, dish, or flask). In some embodiments, the pH of the media is maintained at about 7 to about 9, about 7.5 to about 8.5, about 8, about 7.8 to about 8.2, or 8.

The harvesting of the particles can be done at any time after the nucleic acid encoding the replicon is contacted with the cell that is able to produce the RVPs after being contacted with the replicon. In some embodiments, the RVPs are harvested every 24 hours or at times 72-148 hours post-transfection. In some embodiments the RVPs are harvested every 6 to 8 hours. The RVPs can be harvested by collecting the supernatant of the media that the cells are growing in. The RVPs are then isolated from the media. Any method of isolation can be used to isolate or purify the RVPs away from the media. Examples of isolation and purification include, but are not limited to, filtering the cell media supernatant.

As described herein and above, the present invention provides a cell or “producer cell” that expresses the structural proteins of Dengue virus. The present invention also provides cells comprising the structural proteins of Dengue (C, prM, E). In some embodiments, the cell comprising the structural proteins of Dengue does not comprise the non-structural proteins of Dengue. As used herein, when a cell is referred to as “comprising” a protein it can refer to a cell that is stably transfected and, therefore, stably expresses the protein(s) referred to or it can refer to a cell that is only transiently expressing the proteins. In some embodiments, the cell comprises (e.g. expresses) structural proteins of DEN that include, but are not limited to, C, prM, E, or combinations thereof.

In some embodiments, the cell comprises an inducible promoter controlling the expression of said structural proteins. In some embodiments, the structural genes and/or the inducible promoter are stably integrated into the cell. In some embodiments, the cell comprising the structural proteins of Dengue does not comprise the 5′ untranslated region of Dengue. In some embodiments, the 5′ untranslated region of DEN includes any RNA sequence prior to the first ATG of DEN. In some embodiments, the cell is free of 5′ UTR of DEN upstream of the ATG start codon of the DEN polyprotein comprising the secondary structure that influences translation of the polyprotein and/or the replication of the viral RNA genome.

The structural proteins can be expressed from one or more nucleic acid molecules. In some embodiments, the structural proteins are expressed from a single nucleic acid molecule. In some embodiments, the structural proteins that are expressed from a single nucleic acid molecule are under the control of one or more promoters. In some embodiments, a different promoter can control the expression of each protein, or a first promoter can control the expression of one structural protein and a second promoter can control the expression of the other structural proteins. For example, C, prM, and E can all be controlled by one promoter, or a first promoter can control the expression of C, while a second promoter controls the expression of prM and E. Another example includes, a first promoter operably linked to a nucleic acid molecule encoding the C protein, a second promoter operably linked to a nucleic acid molecule encoding the prM protein, and a third promoter operably linked to a nucleic acid molecule encoding the E protein.

In some embodiments, the nucleic acid molecule encoding the structural proteins is a stable integration. As used herein, “stable integration” refers to any non-endogenous nucleic acid molecule that has been taken up by a cell and has been integrated into the cell genome. Cells comprising a stable integration naturally replicate their genome with the integrated nucleic acid and pass the nucleic acid to daughter cells.

In some embodiments, the nucleic acid molecule encoding the structural proteins is a plasmid. In some embodiments, a cell comprising one or more nucleic acid molecules encoding for the structural proteins is a HEK-293 cell or a cell derived from a HEK-293 cell. As used herein, “a cell derived from a HEK-293 cell” is one where the HEK-293 is the parental cell line and has been modified in such a manner by either recombinant or other techniques such that it is no longer a “wild-type” HEK-293 cell.

As discussed above, the structural proteins can be under the control of one or more inducible promoters and thus one can regulate the production of RVPs. In some embodiments, methods of producing Dengue RVPs comprise contacting a cell with a nucleic acid encoding a Dengue replicon wherein the nucleic acid further comprises nucleic acids encoding the structural proteins of Dengue virus and an inducible promoter which controls expression of said nucleic acids encoding the structural proteins of Dengue virus. Upon contacting the cell with the nucleic acid encoding the replicon the structural proteins are induced. The induction of the expression of the structural proteins along with the presence of the replicon and the expression of the Dengue proteins from the replicon will result in the cell producing Dengue RVPs.

The present invention also provides compositions comprising Dengue reporter virus particles and a storage buffer. The storage buffer is any buffer that allows the Dengue reporter virus particles to be stored (e.g. frozen or refrigerated) for a period of time and the Dengue reporter virus particles maintain their ability to infect Dengue virus susceptible cell (e.g. a cell that can be infected by Dengue virus or RVP). In some embodiments, the storage buffer is maintained at a pH of about 7.5 to about 8.5. In some embodiments, the pH of the storage buffer is 8. In some embodiments the storage buffer is Hepes buffer. In some embodiments, the concentration of HEPES is more than 10 mM. In some embodiments, the concentration of Hepes is 25 mM and/or has a pH of 7.5 to 8.5 or 8. In some embodiments, the storage buffer comprises an additive. As used herein, an “additive” may be any molecule that, when added to a storage buffer comprising RVPs, prevents degradation of RVPs. Examples of additives include, but are not limited to, bovine serum albumin (BSA), fetal calf serum, sugars, or combinations thereof. In some embodiments, the additive must be above a certain concentration in a weight/volume ratio. For instance, in some embodiments, the additive comprises 1% to 10%, 2% to 8%, 3% to 7%, 4% to 6%, or 5% D-Lactose per 100 mL of storage buffer. In some embodiments, the storage buffer comprises a protein additive. In some embodiments, the protein additive must be above a certain concentration in a volume/volume ratio. For instance, in some embodiments, the storage buffer comprises a protein additive at concentrations of 5% to 50%, 15% to 25%, or 20% fetal calf serum. In some embodiments, the total protein additive concentration of the storage buffer is at least 8 μg per mL of storage buffer upon addition of said protein additive.

The present invention also provides methods of infecting a cell with a RVP comprising contacting a cell with a Dengue reporter virus particle. In some embodiments, the cell expresses DC-SIGNR. In some embodiments, the cell that expresses DC-SIGNR is a Raji-DC-SIGNR cell. In some embodiments the cell is a C636 cell or a K562 cell In some embodiments, the RVP is contacted with the cell in the presence of fetal calf serum in the media. In some embodiments, the media comprises about 0.1% to about 10%, about 0.3% to about 3.0%, or about 0.5%, or 0.5% fetal calf serum.

The present invention also provides a method of identifying a compound that can inhibit Dengue infection. In some embodiments, the method comprises contacting a cell with a Dengue RVP in the presence or absence of a test compound and determining if the Dengue RVP can infect said cell in the presence and absence of said test compound. If the Dengue RVP can infect the cell in the absence of the test compound, but not in the presence of the test compound that can inhibit Dengue infection, the test compound is said to be a compound that inhibits Dengue infection. The test compound that can inhibit Dengue infection can be any type of compound or molecule including, but not limited to, a small organic molecule, small peptides, fusions of organic molecules and peptides, and the like. In this particular method, the compound that can inhibit Dengue infection does not include-neutralizing antibodies. Infection can be measured or determined by any manner, but can be for example determined by measuring the expression of the reporter element in the cell. For example, if a Dengue RVP comprises a GFP reporter, the ability to infect a cell can be determined by detecting the expression of GFP in the cell after being contacted with the RVP in the presence or absence of the test compound. If the test compound that can inhibit Dengue infection is a compound that can inhibit the ability of the RVP to infect the cell, the GFP expression will be less than the GFP expression in the absence of the test compound.

The present invention also provides methods of identifying a compound that inhibits Dengue assembly comprising contacting a Dengue RVP producer cell with a test compound and determining if the Dengue RVPs can assemble in the presence of said test compound. A compound that inhibits Dengue assembly can be any compound including but not necessarily limited to small organic compounds, peptides, complete antibodies, any portion of antibody, or fusion compounds of any combination thereof. If assembly is prevented in the presence of the test compound as compared the assembly in the absence of the test compound, the test compound is said to be a compound that inhibits Dengue assembly. A Dengue RVP producer cell is a cell that is capable of producing Dengue RVPs. Producer cells can be generated in any manner including the methods described herein. For example, the method can comprise transfecting the producer with a nucleic acid molecule encoding a Dengue replicon. Assembly can be measured by any manner including measuring the expression of the reporter construct that is part of the RVP, such as, but not limited to, GFP expression. Assembly can also be measured by detecting reporter virus and/or subviral particles in the media by detection of E protein, for example using an ELISA or western blot.

The present invention also provides methods of identifying a compound that inhibits Dengue RNA replication comprising contacting a cell comprising a Dengue replicon with a test compound and measuring Dengue RNA replication, wherein a decrease in Dengue RNA replication indicates that said test compound is a compound that inhibits Dengue RNA replication. A compound that inhibits Dengue RNA replication can be any compound including but not necessarily limited to small organic compounds, peptides, complete antibodies, any portion of antibody, or fusion compounds of any combination thereof. RNA replication can be measured by any method, but can also be determined by measuring the RNA replication or expression of the reporter element by the replicon. For example, if the replicon comprises a GFP reporter, the GFP expression in the cell can be measured to determine if the test compound inhibits RNA replication.

The present invention also provides methods of identifying neutralizing antibodies against Dengue virus. In some embodiments, the method comprises contacting a Dengue RVP with a test antibody; contacting the mixture of the RVP and the test antibody with a cell that can be infected with the RVP in the absence of the test antibody; and measuring the infection of said cell in the presence of said test antibody. If the ability of the RVP to infect the cell is decreased in the presence of the test antibody as compared to when the antibody is not present, this indicates that the test antibody is a neutralizing antibody against Dengue virus. As discussed above, RVP infection can be determined by any method, including, but not limited to measuring reporter expression after infection in the cells. In some embodiments, the reporter is GFP. The test antibody can be any type of antibody including monoclonal antibodies, polyclonal antibodies, antibody fragments, single chain antibodies, scFV, and the like. The antibodies can also be humanized antibodies. The antibodies can also be antibodies from an individual's sera or isolated from an individual.

In some embodiments, the test antibody is a serotype-specific Dengue antibody and the Dengue RVP is a serotype-specific Dengue reporter virus particle. Dengue virus has four serotypes (Dengue 1, Dengue 2, Dengue 3, and Dengue 4). The serotypes can be any of the four serotypes of Dengue virus or any future serotypes of Dengue that are identified. The present invention can also be used to identify serotype-specific neutralizing antibodies by monitoring the association between a test antibody against a serotype-specific Dengue RVP. If a test antibody is a neutralizing antibody against one serotype, but not another, it is said to be specific to at least one serotype DEN virus. A neutralizing antibody can also be neutralizing for more than one serotype but not for all serotypes and such neutralizing antibodies can be identified using the methods described herein.

The invention is now described with reference to the following examples. These examples are provided for the purpose of illustration only and the invention should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLE 1

### Construction of a Monocystronic DNA-Launched Dengue Subgenomic Replicon

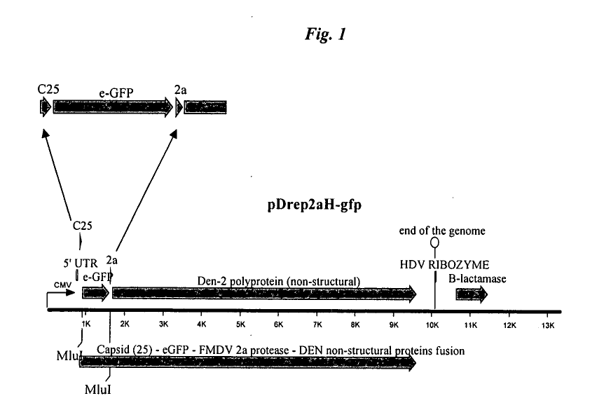

We used a partially constructed Dengue 2 monocystronic replicon plasmid (pDENRep mono 1) as a starting material for constructing a vector useful for RVP replicon production (Holden, et al. (2006), Virology, 344:439-52). This plasmid contained the DEN2 strain 16681 genomic sequence, modified by replacement of a portion of the structural gene sequences with the firefly luciferase gene. Further modification of this plasmid is summarized in FIG. 1.

(i) This vector was first modified by addition of the CMV promoter and FMDV 2a protease using overlapping PCR technology. A fragment of pWrep2aH-(Mlu) (Pierson, et al. (2005), Virology) was amplified with primers WDI (TTTTTTCCAAAGCTATGGTCAATATTGGCCATTAGCCATATTATT) (SEQ ID NO:1) and WD2 (GCTGCGTGAATTCATTCCTATAGGACCAGGGTTACTTTCAAC) (SEQ ID NO:2) using Invitrogen's Platinum Pfx polymerase under the manufacturer's recommended conditions. This fragment carries the CMV promoter, WNV 5′UTR, 20 amino acids of WNV capsid, a unique MluI site, and the FMDV2a autoprotease. Next, a fragment was amplified from pDEN Rep Mono1 using primers WD3 (GTTGAAAGTAACCCTGGTCCTATAGGAATGAATTCACGCAGC) (SEQ ID NO:3) and WD4 (CCACAGGTACCATGCTGCTGCCGTGATTGGTAT) (SEQ ID NO: 4), resulting in a fragment encompassing portions of the DEN E protein and non-structural genes. These two fragments were then used as templates for the overlapping PCR reaction with primers WD1 and WD4, which was digested with BstXI and KpnI, and ligated into the SacI/KpnI sites of pDEN Rep mono 1, resulting in pWDR2A.

(ii) Plasmid pWDR2A was next modified to remove the West Nile sequences. Plasmid pDEN Rep Mono1 was used as template for amplification of the Dengue 5′ UTR and capsid sequences using primers DR1 (TTTTTCAGAGCTCGTTTAGTGAACCGAGTTGTTAGTCTACGTGG ACCGAC) (SEQ ID NO: 5) and DR2 (AAGTTACGCGTGGACACGCGGTTTCTCTCGCG) (SEQ ID NO: 6). The resulting fragment was digested with MluI and SacI, and then ligated into pWDR2A using those same sites, generating pDR2A.

(iii) pDR2A was then modified by addition of a HDV ribozyme and poly A tail cassette into the 3′ end of the genome. First, primers Drep1 (GAAGCCCTAGGATTCTTAAATGAAGAT) (SEQ ID NO: 7) and Drep2 (AGGCTGGGACCATGCCGGCCAGAACCTGTTGATTCAACAGCA) (SEQ ID NO: 8) were used to amplify a fragment from pDR2A. The HDVR was amplified by primers Drep3 (TGCTGTTGAATCAACAGGTTCT GGCCGGCATGGTCCCAGCCT) (SEQ ID NO: 9) and Drep4 (TTATCATCGATTACCACATTTGTAGAGGTTTTACTTGC) (SEQ ID NO: 10) using plasmid pWrep2aH-(Mlu) for template. Both fragments were then used as template with primers Drep1 and Drep4 to generate a PCR fragment containing the 3′ end of the DEN2 genome fused to the HDVR. This fragment was digested with AvrII and ClaI, then inserted into the same sites in pDR2A, resulting in plasmid pDR2AH.

(iv) The plasmid was further modified to insert a reporter gene. The eGFP sequence was digested from pWNII Rep2AH eGFP using the MluI restriction enzyme. This fragment was ligated into the unique MluI site of pDR2AH.

Three additional replicons were constructed for the expression of Renilla luciferase (pDR2AH Ren), a Renilla Zeocin resistance fusion protein (pDR2AH RenZeo), and e-GFP Zeocin resistance fusion protein (pDR2AH GFPZeo). Renilla luciferase was amplified from a pRL-TK (Promega, using primers Ren 5′ AAAAAAACGCGTATGGCTTCGAAAGTTTATGATCCAGAA (SEQ ID NO: 11) and Ren 3′

| | | | |
|---|---|---|---|
| Primer | | | |
| Name | Recognizes | Primer Sequence | SEQ ID NO: |

AAAAAAGGCGCGCCGTGATAGATCTTTGTTCATTTTTGAG (SEQ ID NO: 12). The PCR product was digested with MluI and AscI, then ligated into the MluI site of pDR2AH, resulting in pDR2AH Renilla. The RenZeo fusion was generated using overlapping PCR. The following oligos were used for cloning:

TABLE 1
Primer Sequences Used for Generation of RenZeo fusion construct.

| | | | |
|---|---|---|---|
| d27 | GFP forward | AAAAAAACGCGTATGGTGAGCAAGGGCGAGGAGCTGTTCA | SEQ ID NO:13 |
| d28 | Zeo Reverse | TTTTTACGCGTGTCCTGCTCCTCGGCCACGAAGTGCA | SEQ ID NO:14 |
| d29 | (zeo) GFP | TTGGCCTTGTACAGCTCGTCCATGCCGAGAGTGA | SEQ ID NO:15 |
| | reverse | | |
| d30 | (gfp) Zeo | ACAAGGCCAAGTTGACCAGTGCCGTTCCGGTGCTCA | SEQ ID NO:16 |
| | forward | | |
| d31 | Renilla | AAAAAAACGCGTATGACTTCGAAAGTTTATGATCCAGAAC | SEQ ID NO:17 |
| | forward | | |
| d32 | (zeo) | TTGGCTTGTTCATTTTTGAGAACTCGCTCAACGAACG | SEQ ID NO:18 |
| | Renilla | | |
| | reverse | | |
| d33 | (ren) Zeo | AACAAGCCAAGTTGACCAGTGCCGTTCCGGTGCTCACCGC | SEQ ID NO:19 |
| | forward | | |

To generate the GFP-zeocin fusion product, eGFP was amplified from pDR2AH GFP using primers d27 and d29, and zeocin was amplified from plasmid pcDNA3.1+Zeo with primers d30 and d28. These products were resolved on an agarose gel, extracted, and used as template for PCR with primers d27 and d28. This product was resolved on an agarose gel, extracted, and then digested with MluI and inserted into the MluI site of pDR2AH, resulting in plasmid pDR2AH GFP Zeo.

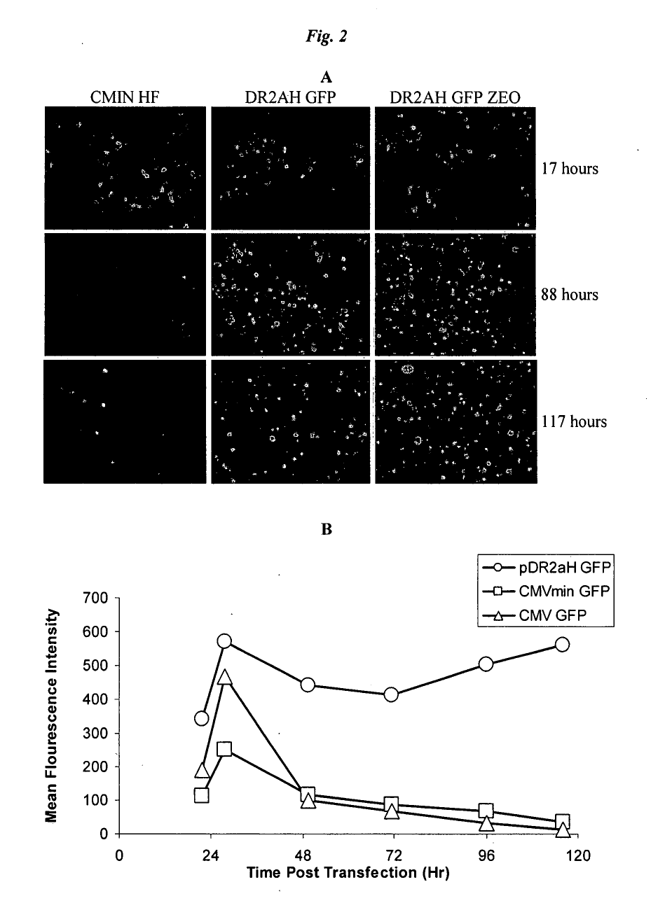

Plasmid pDR2AH Ren Zeo was generated by PCR amplication of the gene from pDR2AH Renilla using primers d31 and d32. The zeocin fragment was amplified from plasmid pcDNA3.1+Zeo with primers d32 and d28. These two products were resolved on an agarose gel, extracted, and used as template for amplification with primers d31 and d28. The resulting product was resolved on an agarose gel, extracted, digested with MluI, and inserted into the MluI site of pDR2AH, resulting in plasmid pDR2AH RenZeo. Plasmids pGFP, pDR2AH GFP, pDR2AH Ren, pDR2AH GFP ZEO, and pDR2AH RenZeo were transfected into 293T cells using a standard calcium phosphate transfection method. Cell culture media was changed approximate 4-16 hours post-transfection. Cells transfected with GFP encoding replicons were examined for GFP expression visually using a fluorescent microscope. Both replicon pDR2AH GFP and pDR2AH GFPZeo resulted in GFP expression in target cells (FIG. 2, Panel A). Cells transfected with pGFP or pDR2AH GFP were analyzed by flow cytometry at multiple time points post-transfection. Prolonged GFP expression was observed in pDR2AH GFP replicon transfected cells days after it was no longer observed in pGFP plasmid transfected cells (FIG. 2, Panel B), indicating productive replication of the GFP replicon within the cells.

### EXAMPLE 2

### Construction of a Plasmid that Expresses DEN Structural Proteins

Molecular clones for the WestPac Dengue 1, and PDK50, New Guinea C, and 16681 Dengue 2 strains were obtained. These constructs were used as templates for amplification of the capsid, premembrane protein, and envelope structural gene regions. Primers used for the reactions are listed in Table 2 below:

TABLE 2
Primers used to construct Sequence #1

| | |
|---|---|
| Primer | |
| Name | Primer Sequence |

| | | |
|---|---|---|
| d 1 | caccATGAATAACCAACGGAAAAAGGCGA | (SEQ ID NO: 20) |
| d 2 | TTTCACTATTAGGCCTGCACCATGACTCCCAAATAC | (SEQ ID NO: 21) |
| d 3 | caccATGAACAACCAACGGAAAAAGACGGGT | (SEQ ID NO: 22) |
| d 4 | TTTCACTATTACGCCTGAACCATGACTCCTAGGTAC | (SEQ ID NO: 23) |
| DR2 | AAGTTACGCGTGGACACGCGGTTTCTCTCGCG | (SEQ ID NO: 6) |
| DREP1 | GAAGCCCTAGGATTCTTAAATGAAGAT | (SEQ ID NO: 7) |
| DREP4 | TTATCATCGATTACCACATTTGTAGAGGTTTTACTTGC | (SEQ ID NO: 10) |
| WD1 | TTTTTTCCAAAGCTATGGTCAATATTGGCCATTAGCCATATTATT | (SEQ ID NO: 1) |
| WD2 | GCTGCGTGAATTCATTCCTATAGGACCAGGGTTACTTTCAAC | (SEQ ID NO: 2) |
| DREP2 | GCTGCGTGAATTCATTCCTATAGGACCAGGGTTACTTTCAAC | (SEQ ID NO: 8) |
| DREP3 | TGCTGTTGAATCAACAGGTTCTGGCCGGCATGGTCCCAGCCT | (SEQ ID NO: 9) |
| DR1 | TTTTTCAGAGCTCGTTTAGTGAACCGAGTTGTTAGTCTACGTGGACCGAC | (SEQ ID NO: 5) |
| WD3 | GTTGAAAGTAACCCTGGTCCTATAGGAATGAATTCACGCAGC | (SEQ ID NO: 3) |
| WD4 | CCACAGGTACCATGCTGCTGCCGTGATTGGTAT | (SEQ ID NO: 4) |
| d 5 | caccATGTCTGCAGGCATGATCATTATGC | (SEQ ID NO: 24) |
| d 6 | CACCATGTCTGTGACCATGCTCCTCATGCT | (SEQ ID NO: 25) |

The WestPac CME fragment was amplified using primers d3 and d4, while New Guinea C, S16803, and 16681 were amplified with primers d1 and d2. PCR products were generated using Invitrogen's Platinum Pfx polymerase under recommended conditions. PCR reactions were then loaded on a 1.5% agarose gel and resolved by electrophoresis. Resulting 2.3 kb bands were extracted from agarose using the Qiagen Gel Extraction kit and eluted with water. Fragments were ligated into pENTR/D topo as recommended by the manufacturer, and then transformed into STBL2 competent cells and cultured at 30° C. Positive clones were identified by restriction digestion and sequences verified by dye terminator sequencing. Correct clones were then used to generate expression constructs in the pcDNA6.2-DEST and pT-Rex-DEST30 vectors. Recombination reactions were performed using LR Clonase according to the manufacturer's directions. Resulting clones were screened by ampicillin resistance and restriction digestions to verify the plasmid and insert. All plasmid preparations were cultured using STBL2 bacteria, 30° C. incubation temperature, and 50 μg/ml ampicillin selection agent. DEN structural gene expression was confirmed by Lipofectamine transfection of BHK-21 clone 15 cells followed by immunofluorescent staining with mAb 4G2, 2H2, and/or 3H5. Transfected cells were fixed with cold methanol, washed with PBS, and then probed with primary antibody for 1 hour on ice. Cells were then washed with PBS and then incubated with a Cy3-conjugated anti-mouse secondary antibody for one hour on ice. Cells were then washed again with PBS and examined with an inverted fluorescent microscope. All cells expressed the expected proteins.

Additional DEN sequences were also prepared. The prME sequences were PCR amplified from plasmids encoding the 16681, S16803, NGC, and WestPac dengue strains. Primers used for amplification of the DEN 2 sequences (16681, NGC, S16803) were d5 and d2. DEN 1 prME sequence from WestPac was amplified using primers d6 and d4. PCR products were amplified using the Invitrogen Platinum Pfx polymerase under standard conditions. Reactions were analyzed by gel electrophoresis and the resulting bands isolated, extracted from agarose, and cloned into pENTR/D Topo as recommended by the manufacturer. Inserts were confirmed by restriction digestion and dye terminator sequencing. Correct clones were then used to generate expression constructs in the pcDNA6.2-DEST and pT-Rex-DEST30 vectors. Recombination reactions were performed using LR Clonase according to the manufacturer's directions. Resulting clones were screened by ampicillin resistance and restriction digestions to verify the presence of the desired insert. All plasmid preparations were cultured using STBL2 bacteria, 30° C. incubation temperature, and 50 μg/ml ampicillin selection agent.

### EXAMPLE 3

### Construction of Cell Lines for Producing DEN Reporter Virus and SVPs

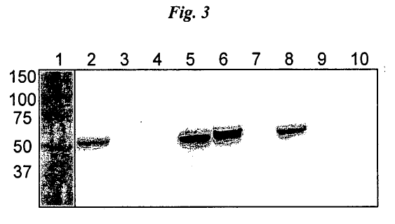

Cell lines were generated using the Invitrogen T-REx 293 cell line. Cells were plated in 6 well dishes at one million cells per well in complete medium. Plasmids containing DEN structural proteins (prME) from different strains, as described herein, were used. Plasmids were transfected using Lipofectamine 2000 as recommended by the manufacturer. 48 hours post-transfection, cells were trypsinized and plated in T75 flasks with complete DMEM, supplemented with 500 μg/ml G418 and 10 μg/ml blasticidin. Cells were cultured for three weeks in selective media with trypsinization and reseeding as needed upon reaching near confluence. Pooled selected cells were aliquotted and frozen in FCS, 10% DMSO freezing medium and placed in liquid nitrogen storage. Cells were split into 6 well dishes and cultured in the presence or absence of 1 μg/ml doxycycline. Cells were lysed with PBS, 0.5% Triton X-100, and soluble lysate examined for CME expression by electrophoresis on an 8-16% acrylamide gel and western blotting with antibodies 4G2 and 2H2 (FIG. 3). CME protein was observed only upon addition of doxycycline to the culture medium.

### EXAMPLE 4

### Production of DEN Reporter Virus Particles

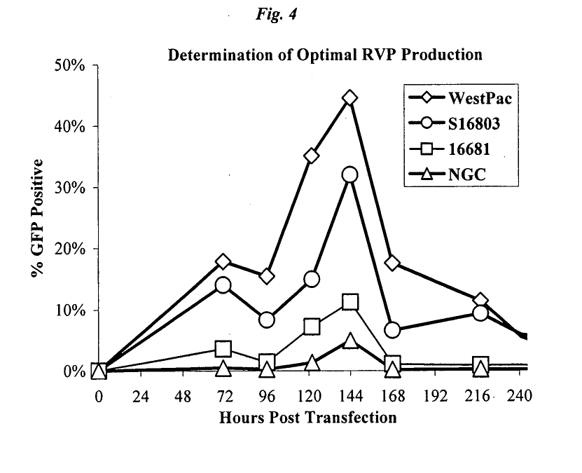

DEN reporter virus particles (RVPs) were produced by calcium phosphate transfection of replicon plasmid into a 50-60% confluent CME 293trx stable line. 4-16 hours post-transfection, the media was replaced with RVP production media (DMEM-10% FCS, 1% penicillin/streptomycin solution, 2 mM L-alanyl L-glutamine solution, 25 mM HEPES, 1 μg/ml doxycycline, pH 8.0). Harvests of RVPs from the producer cells were taken at 72 hours post-transfection, and repeated every 24 hours up to 7 days post-transfection. Harvested supernatants were filtered through 0.45 μm syringe filter units and placed at 4° C. until needed. RVPs were then used to infect Raji-DC-SIGN-R cells permissive for Dengue replication. 48 hours after infection, cells were transferred to a 96-well cluster tube plate and fixed with paraformaldehyde. Fixed cells were then analyzed by flow cytometry to determine the percent of cells expressing GFP (FIG. 4). RVP supernatants were supplemented with 25 mM HEPES, pH 8, fetal bovine serum, 2.5% or 5% D-lactose or D-glucose, and subsequently frozen by submersion in a dry ice-ethanol bath, placed in a −80° C. freezer for storage, and tested 24 hours, 2 weeks, and 5 months post-freezing for infectivity of Raji-DC-SIGN-R cells. Frozen RVPs retained 50-90% of infectivity as compared to RVPs tested immediately after production.

### EXAMPLE 5

### Storage of DEN Reporter Virus Particles

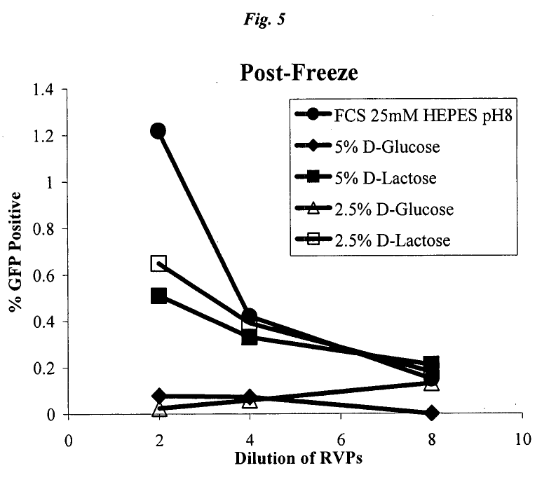

DEN RVPs were produced by transfection of stable cell lines carrying the CprME coding sequence of WP, NGC, 16681, or S168003 Dengue strains under a tetracycline inducible promoter. Cells were induced with doxycycline (1 ug/ml) 4-12 hours post-transfection, and cultured in RVP harvest medium. Supernatants were harvested at various timepoints post-transfection and filtered through 0.45 um syringe filter units. RVPs were then aliquotted into 2 ml screwcap tubes on ice and supplemented with lactose or glucose pH 8.0 (final 5%). Vials were submerged in a pre-cooled dry ice/ethanol bath until frozen and then moved to storage in a −80° C. freezer. Twenty four hours after freezing, the samples were rapidly thawed in a 37° C. water bath and then used to infect Raji-DC-SIGNR cells. Approximately 48 hours post infection, Raji-DC-SIGNR cells were fixed with paraformaldehyde (2% final) and analyzed by flow cytometry to determine the percentage expressing GFP (FIG. 5).

### EXAMPLE 6

### Use of DEN Reporter Virus Particles on Multiple Cell Types

RVPs were either freshly harvested or rapidly thawed from cryopreservation then placed on ice until use. Target cells were counted and plated as follows:
BHK-21 clone 15 cells: 30,000 cells per well in a 24-well plate
Vero cells: 20,000 cells per well in a 24-well plate
Raji DC-SIGN-R cells: 30,000 cells per well in a 96-well plate.

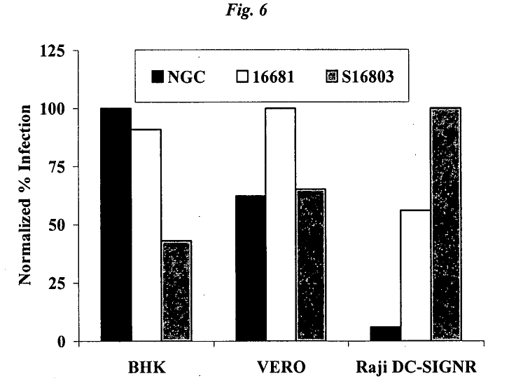

RVPS were diluted with DMEM-10% FCS, 1% penicillin streptomycin, 2 mM L-Alanyl-L-glutamine dipeptide. Aliquots of each dilution were added to target cells and cells were returned to a 37° C., 5% CO₂ incubator for 36-48 hours. Cells were fixed with paraformaldehyde and analyzed by flow cytometry to determine the percentage of cells expressing GFP (FIG. 6).

### EXAMPLE 7

### Use of DEN Reporter Virus Particles to Detect Neutralizing Antibodies

Hybridomas secreting the monoclonal antibodies 4G2, 3H5, 2H2, 1H10, 5D4, and 15F3 were obtained from the ATCC. Cells were cultured in Hybridoma-SFM culture medium (Invitrogen) supplemented with 0.5% Penn-Strep solution. Hybridoma supernatants were collected, filtered through 0.22 μm filters, and then purified using protein A or protein G affinity chromatography. Purified antibodies were dialyzed into PBS, quantitated with a BCA protein assay (Pierce), aliquoted, and stored at −80° C. until needed.

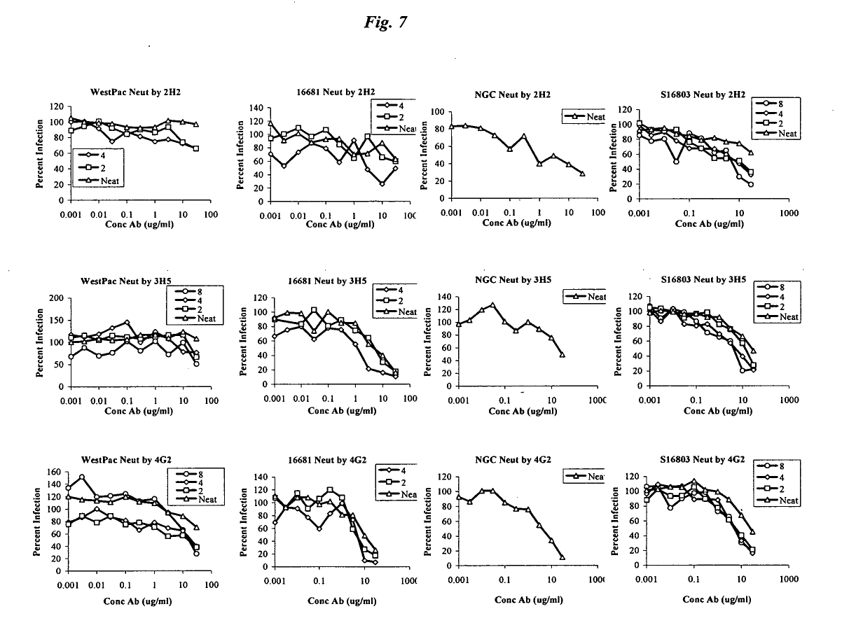

Monoclonal antibodies 2H2, 3H5, and 4G2 were diluted into media (RPMI-10% FCS, 1% penn/strep, 2 mM L-alanyl-1-glutamine, 25 mM HEPES, pH 8) to a final concentration of 60 μg per mL and filter sterilized through a 0.22 um syringe filter. Serial half-log dilutions of antibody were generated, and then 100 ul aliquots were transferred to a 96-well plate. RVPs from DEN strains WestPac, 16681, New Guinea C (NGC), and S168003 were then added directly to the 96-well plate and incubated at room temp with shaking for one hour. Raji DC-SIGN-R cells were counted and resuspended at a concentration of 3 million cells per mL, then added to the wells in 10 ul aliquots. Approximately 48 hours after infection, the cells were fixed with 2% paraformaldehyde and analyzed by flow cytometry for the percentage of GFP positive cells (FIG. 7).

### EXAMPLE 8

### DEN Reporter Virus Particles can be Used to Detect Neutralizing Antibodies in Serum

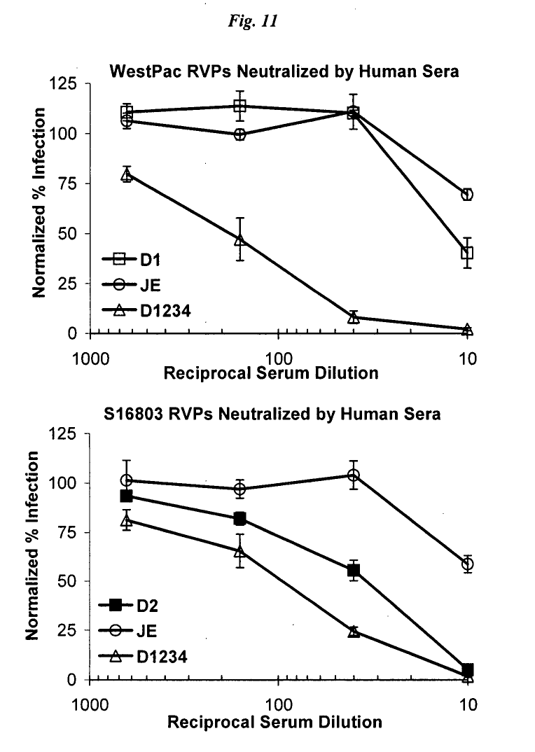

The use of Dengue Reporter Virus Particles to monitor the occurrence of neutralizing antibodies in sera has utility for a number of applications, including vaccine trials. Convalescent sera from naturally infected individuals were obtained from the UK National Institute for Biological Standards and Controls (NIBSC), and complement was heat-inactivated at 56° C. for 30 minutes. Serum precipitates were removed by brief centrifugation in a microcentrifuge, and the supernatant transferred to a sterile tube. Clarified sera were serially diluted from 1:5 to 1:320 in RPMI medium (5% FCS, 1% penn-strep, 25 mM HEPES, 1% L-alanyl-L-glutamine dipeptide solution, pH 8), and 90 μl aliquots of each dilution, as well as a no serum control, transferred to a 96-well plate. RVPs (3×10⁴ infectious units per ml), generated as described herein from Westpac or S16803 strains, were added to each well, and the plate was slowly agitated for one hour at room temperature. Raji-DC-SIGNR cells were then added at 30,000 cells per well, and the plate was incubated at 37° C., 5% CO₂. After 36-48 hours the cells were examined for GFP expression by flow cytometry analysis, using a Guava Easycyte. Neutralization of RVPs, quantified by calculating the ratio of infected (GFP-positive) cells in each well to those in the control (no sera) wells, was correlated with the concentration of immune serum in each well (FIG. 11). These data indicate that RVPs, constructed using diverse Dengue strains, are capable of quantifiably monitoring the presence of neutralizing antibodies in serum.

### EXAMPLE 9

### Use of DEN Reporter Virus Particles to Detect a Drug that Inhibits Entry

Raji-DC-Sign R cells will be cultured in a 96-well plates at 30,000 cells per well. An equal volume of drug (or no drug control) will be added to the well at a 3× concentration before dilution with cells and RVPs (1× final in each well). An equal volume of RVPs are then added to the well and the plate will be returned to a 37° C., 5% CO2 incubator for 36-48 hours. Cells will then be analyzed for GFP expression by flow cytometry and percent GFP-positive cells determined. The extent of inhibition is calculated by dividing the (percent GFP-positive cells contacted drug) value by the (percent GFP-positive cells unexposed to drug) value. Other microplate formats (e.g. 384-well, 1536-well) could also be used. Other orders of addition of the cells and/or RVPs could also be performed.

### EXAMPLE 10

### Use of DEN Reporter Virus Particles to Detect a Drug that Inhibits Assembly

CME expression cell lines will be cultured in 96-well plates. Cells will be transfected with replicon plasmid using calcium phosphate transfection or Lipofectamine transfection. Cells may be transfected either before or after addition to the microplates. At 72 hours post-transfection, the media will be changed on the cells to complete DMEM, pH 8 containing doxycycline and a different inhibitor in each well. Supernatants will be harvested from the producer cells at 96 hours post-transfection, frozen briefly, and then used to infect RajiDC-SIGNR cells. Approximately 48 hours after infection, the cells are fixed with 2% paraformaldehyde and analyzed by flow cytometry for the percentage of GFP positive cells.

### EXAMPLE 11

### Use of DEN Cell Lines Producing SVPs to Detect a Drug that Inhibits Assembly

Cell lines stable for prME will be plated in 96-well plates at a density of 10,000 cells per well. Cells will be cultured in 100 μl media (DMEM-10% FCS, 1% Penicillin-Streptomycin solution, and 2 mM L-Alanyl, L-Glutamine dipeptide solution) overnight. The following day, the media will be supplemented with DMEM-10% FCS, 1% Penicillin-Streptomycin solution, 2 mM L-Alanyl, L-Glutamine dipeptide solution, and 50 mM HEPES, pH 8.0, 2 μg/ml doxycycline and assembly inhibitors (one per well at 1× final in each well). Cells will be incubated overnight, and then media will be assayed by antigen capture assay using monoclonal antibody 2H2 for capture and biotinylated-4G2 for detection. Inhibition of SVP production will be indicated by a loss in signal compared to a control well cultured without assembly inhibitor.

### EXAMPLE 12

### Quantification of DEN E Protein

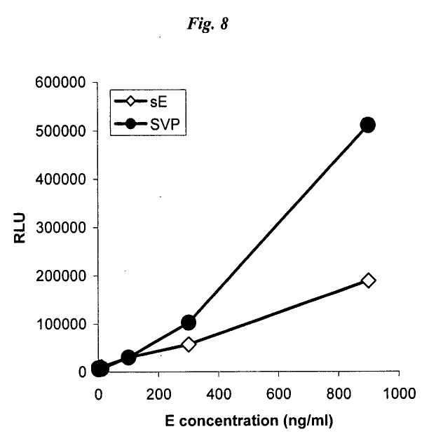

E protein was quantitated using an antigen capture ELISA. The capture and primary antibodies were used in the following combinations: A) Capture with 3H5, detection with biotinylated 4G2. White half-well plates were coated with 50 μl of capture antibody at a final concentration of 10 μg/ml in capture buffer (20 mM Tris pH 8.5, 100 mM NaCl, 0.05% NaN₃). Plates were sealed with tape and incubated at room temp for 2 hours. Wells were then blocked by removal of the capture solution and addition of 100 μl Blotto (2% dry milk, 1×PBS, 0.05% Tween-20) and incubated at room temperature with shaking for 10 minutes. This solution was then removed and replaced with an additional 100 μl Blotto and again incubated for 10 minutes with shaking at room temperature. Samples containing purified SVPs were treated with 0.1% Triton X-100. Two-fold serial dilutions of each sample were made in Blotto. Dilutions were then transferred to the blocked plate in 50 μl aliquots and incubated with shaking for one hour at room temperature. Plates were then washed 3-5 times with wash buffer (PBS, 0.05% Tween). Primary detection antibody (200 ng/ml in Blotto) was then added to each well in a volume of 50 μl and plates were returned to the plate shaker for one hour. The plate was then washed three times with wash buffer, and 50 μl streptavidin HRP (500 ng/ml) diluted in Blotto was added to each well. After one hour incubation with shaking, the washing procedure was repeated, and the plate was developed with 50 μl Supersignal Pico (Pierce, Rockford, Ill.) and read in a Wallac Victor V plate reader. Results are plotted in FIG. 8. One skilled in the art would recognize that other combinations of antibodies and/or sera could also be used for capture and/or detection.

### EXAMPLE 13

### Comparison of RVP Infectivity when Produced at Varying pH

Each CME cell line will be plated in two T75 flasks at a density of 4 million cells per flask. Cells will be cultured overnight in DMEM-10% FCS, 1% Penicillin-Streptomycin solution, 2 mM L-Alanyl, L-Glutamine dipeptide solution, and 10 mM HEPES, pH 7.2. On day 2, replicon-encoding plasmid will be transfected into the cells. Approximately 4 hours after transfection, the media will be replaced in one set of flasks with DMEM-10% FCS, 1% Penicillin-Streptomycin solution, 2 mM L-Alanyl, L-Glutamine dipeptide solution, and 10 mM HEPES, pH 7.2 supplemented with 1 μg/ml doxycycline. In the duplicate set of flasks, the media will be replaced with DMEM-10% FCS, 1% Penicillin-Streptomycin solution, 2 mM L-Alanyl, L-Glutamine dipeptide solution, and 25 mM HEPES, pH 8.0 supplemented with 1 μg/ml doxycycline. At approximately 96 hours after transfection, the cell culture medium will be harvested, filtered through a 0.45 um syringe filter, and then used to infect Raji-DC-SIGN-R cells. Aliquots of RVPs (100 μl) will be placed in a 96-well plate, and four serial two fold dilutions will be generated using RPMI, 10% FCS, 1% Penicillin-streptomycin solution, 25 mM HEPES, pH 7.2. Raji DC-SIGN-R cells will be added in 100 μl aliquots at a density of 300,000 cells per mL. 48 hours after infection, cells will be fixed with 2% paraformaldehyde and quantitated for percentage expressing GFP.

### EXAMPLE 14

### Infectivity of RVPs Produced at pH 8.0, pH 7.2, and Below pH 7

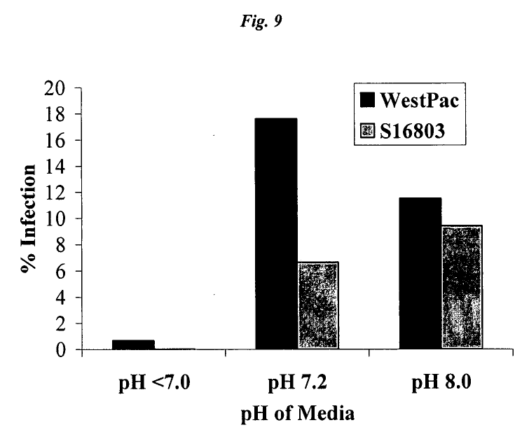

CME expression cell lines (WestPac and S16803) were plated in T75 flasks at a density of 4 million cells per flask and cultured overnight in DMEM-10% FCS, 1% Penicillin-Streptomycin solution, 2 mM L-Alanyl, L-Glutamine dipeptide solution, and 10 mM HEPES, pH 7.2. Each flask was transfected with pDrep2AH GFP plasmid using a standard calcium phosphate protocol. Tissue culture medium was replaced at approximately 18 hours post-transfection with DMEM-10% FCS, 1% Penicillin-Streptomycin solution, 2 mM L-Alanyl, L-Glutamine dipeptide solution, 10 mM HEPES, pH 7.2, and 1 ug/ml doxycycline. At 144 hours post-transfection, the cell culture medium was harvested and replaced with DMEM-10% FCS, 1% Penicillin-Streptomycin solution, 2 mM L-Alanyl, L-Glutamine dipeptide solution, 10 mM HEPES, pH 7.2, and 1 ug/ml doxycycline. RVPs were allowed to accumulate for 24 hours, then harvested, filtered through 0.45 μm syringe filters, and used to infect Raji-DC-SIGN-R cells (“pH 7.2”). The cell culture medium was replaced and media harvest repeated at 192 hours post-transfection. The pH of the media at the 192 h harvest was below pH 7 (“pH<7.0”) due to additional cell growth and metabolism. RVPs were again harvested and used to infect Raji-DC-SIGN-R cells. The cell culture medium was replaced with DMEM-10% FCS, 1% Penicillin-Streptomycin solution, 2 mM L-Alanyl, L-Glutamine dipeptide solution, 25 mM HEPES, pH 8.0, that had been adjusted to a final pH of 8.0. In this case, the HEPES concentration and pH were adjusted in order to increase the buffering of the media during production. Cells were cultured overnight and the medium was harvested again at 218 hours post-transfection and used to infect Raji-DC-SIGN-R cells (“pH 8.0”). Each set of infected Raji DC-SIGN R cells were fixed with 2% paraformaldehyde at 48 hours post-infection, and analyzed by flow cytometry (FIG. 9).

### EXAMPLE 15

### DEN RVPs can be Used to Detect Enhancing Monoclonal Antibodies

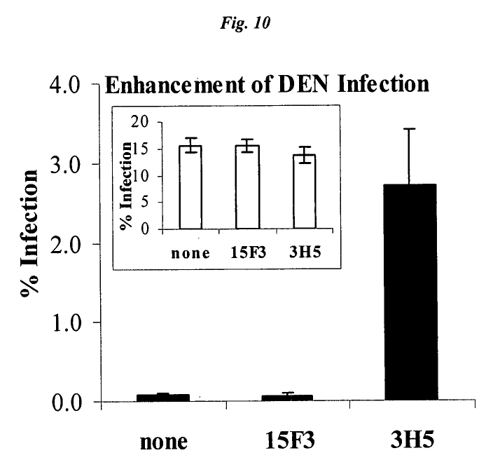

The detection of antibody-mediated enhancement of DEN infection is an important strategy for monitoring dengue epidemiology. Anti-dengue monoclonal antibodies 3H5, 4G2, and 15F3 were diluted to a final concentration of 4 μg per mL using RPMI medium (10% FCS, 1% penn/strep, 2 mM L-alanyl-1-glutamine, 25 mM HEPES, pH 8), 0.22 μm filter sterilized, and aliquotted, in duplicate, into separate wells of a 96-well plate. RVPs (3×10⁴ infectious units per ml), generated from DEN strains WestPac, 16681, and S16803, were then added to each well and incubated at room temperature with shaking for one hour. Fc-receptor positive (K562) or Fc-receptor negative (Raji DC-SIGNR) cells were then added to each well to a final concentration of 30,000 cells per well. Approximately 48 hours later, the cells were fixed with 2% paraformaldehyde, and analyzed by flow cytometry for expression of GFP as an indication of RVP infection. Antibody-mediated enhancement of infection by 3H5 and 4G2 was indicated by a greater percentage of infected K562 cells relative to that obtained in the absence of antibody, or in the presence of a non-specific control antibody (15F3) (FIG. 10 and data not shown). The presence of these monoclonal antibodies either did not influence, or inhibited in a strain-specific manner, the rate of infection of cells (DC-SIGNR) not expressing Fc-receptor. These results indicate that RVPs can specifically detect the presence of infection-enhancing antibodies using appropriate target cells. One skilled in the art would recognize that other Fc-positive cells, including dendritic cells, primary monocytes (Kliks (1990), AIDS Res Hum Retroviruses, 6:993-8, Kliks, et al. (1989), Am J Trop Med Hyg, 40:444-51), and other Fc-positive cell lines, could similarly be used with Dengue RVPs to detect antibody-mediated enhancement.

### EXAMPLE 16

### DEN Reporter Virus Particles can Detect Enhancing Antibodies in Sera

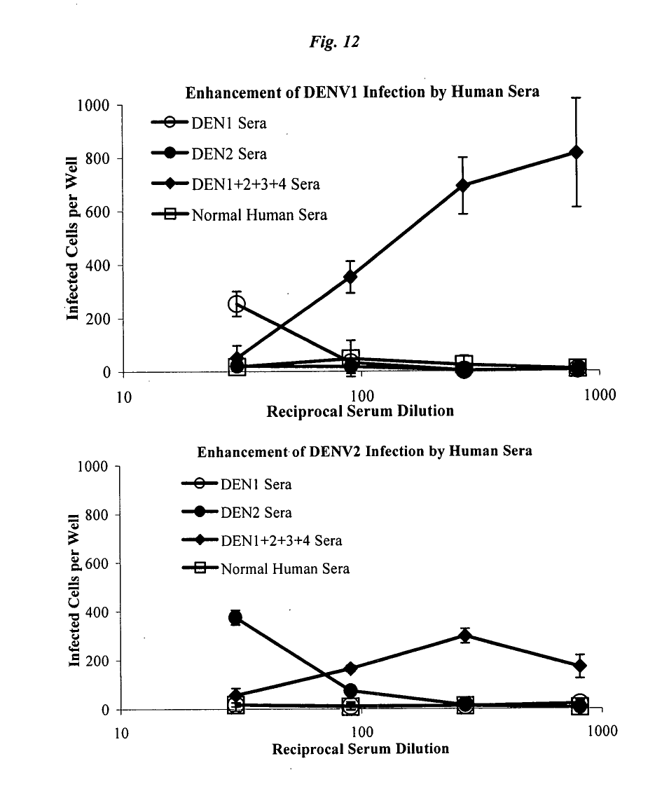

Dengue RVPs can be used to quantifiably detect antibody-mediated enhancement in serum collected from exposed individuals. Dengue convalescent human sera were obtained from the UK National Institute for Biological Standards and Controls (NIBSC). Convalescent serum for DEN1 (NIBSC Code 02/300, DEN2 (NIBSC Code 02/296), negative control (NIBSC Code 02/184) and tetravalent human sera (NIBSC Code 02/186) were diluted with 500 μl sterile water, and complement heat-inactivated at 56° C. for 30 minutes. Serum precipitates were pelleted by brief centrifugation in a microcentrifuge and the supernatant transferred to a sterile tube. Particulates were further removed by passage through a 0.45 μm syringe filter system. Serial 3-fold dilutions of filtered serum supernatants were generated in RPMI medium (5% FCS, 1% penn/strep, 2 mM L-alanyl-1-glutamine, 25 mM HEPES, pH 8). Aliquots (50 μl) of each dilution were transferred into a 96-well plate, mixed with an equal volume of either WestPac or S116803 strain DEN RVPs, and allowed to bind at room temperature for approximately 10 minutes. Fc-receptor positive (K652) target cells were then added to each well to a final concentration 30,000 cells per well. Approximately 48 hours later, cells were fixed with 2% paraformaldehyde, and the number of infected (GFP-positive) cells per well determined by flow cytometry using a Guava Easycyte. Antibody-mediated enhancement of RVP infection, indicated by a greater percentage of infected cells compared with cells infected in the presence of naïve human control serum, was detected in convalescent serum in a strain-specific manner (FIG. 12). No enhancement of DEN RVP infection was observed in the presence of serum from individuals infected with Dengue virus of a different serotype to that from which RVPs were generated (FIG. 12 and data not shown), indicating the specificity with which DEN RVPs can detect antibody-mediated enhancement.

### EXAMPLE 17

### Dengue Reporter Virus Particles Carrying a Renilla luciferase Reporter Gene can be Used to Indicate Cell Infection

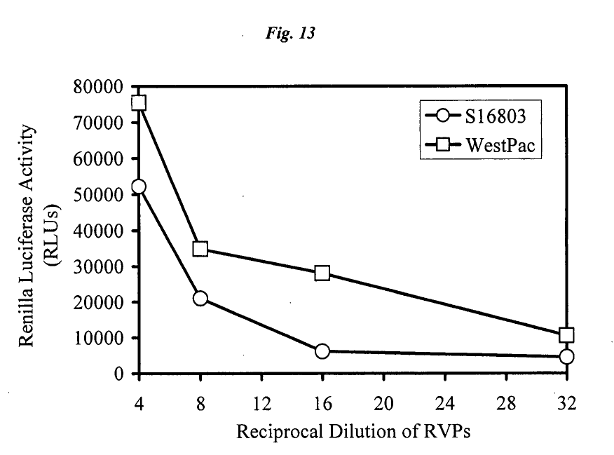

Functional Dengue RVPs can be generated using GFP or a variety of different protein reporters, including Renilla Luciferase, as demonstrated here. Reporter virus particles (Westpac or S16803 strains) were produced and harvested, as described herein, incorporating a Renilla luciferase reporter gene from the sea pansy (Renilla). Briefly, packaging cell lines were transfected with the Dengue Renilla replicon plasmid via calcium phosphate. At 24 hours after transfection, the culture medium was exchanged with complete DMEM, pH of 8.0 supplemented with 25 mM HEPES and 1 μg per ml of doxycycline. RVPs were harvested at 72 hours post-transfection and at 24 hour intervals for the following 5 days. RVPs were diluted 1:1 in RPMI medium (10% FBS, 1% penicillin-streptomycin, 1% L-alanyl-L-glutamine dipeptide solution, 10 mM HEPES, pH 8) and aliquotted in a 96-well plate. Raji DC-SIGNR cells (30,000) were added to each well and the plate was placed in a 37° C. incubator with 5% CO₂. After various time intervals, well contents were transferred to a new 96-well V-bottom plate, and cells harvested by centrifugation. Cell pellets were re-suspended in 30 μl Renilla Lysis buffer (Promega) and shaken for 10 minutes at room temperature. Lysates were transferred to a white 96-well plate, and luciferase expression quantified using a Renilla Luciferase Assay Kit (Promega) and a Wallac Victor V luminometer. Infection of target cells was evident from the expression of the luciferase reporter gene, delivered by RVPs constructed from either DEN1 or DEN2 strains (FIG. 13).

### EXAMPLE 18

### Dengue Reporter Virus Particles can be Produced Using Cloned Cell Lines

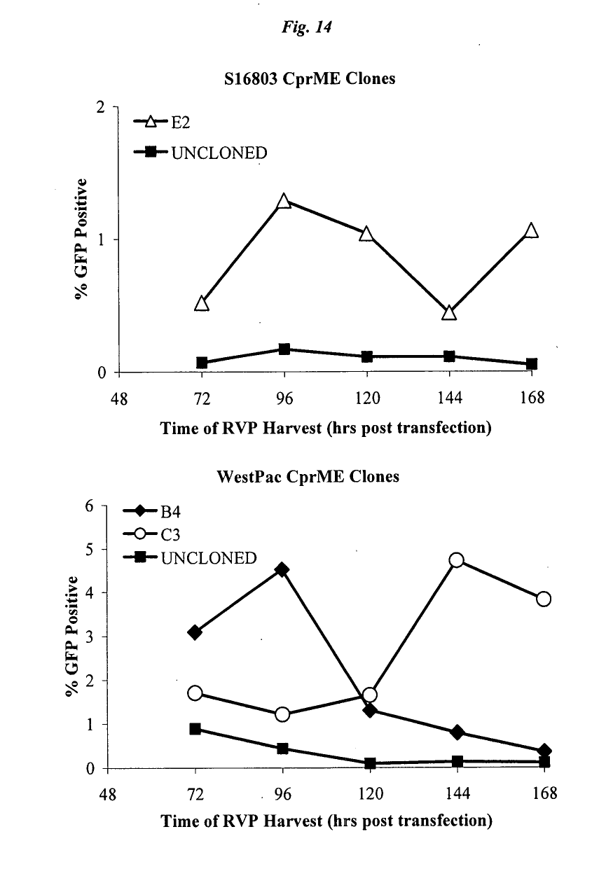

Populations of DEN RVP producer cells (293trx), containing mixed subsets of cells expressing CprME genes at different quantities, can change over time toward poorer RVP-producing cells. Here, we demonstrate that single-population subsets of DEN RVP producer cells, with desirable characteristics, can be cloned from these heterogeneous populations. 293trx cells, produced by stable transfection with the CprME expression plasmid, as described herein, were cloned from pooled producer cell lines using a limiting dilution technique. Briefly, 293trx cells were plated in 96-well plates at approximately 10 cells per well in a volume of 100 μl and cultured. After approximately two weeks of incubation at 37° C., 5% CO₂ wells were examined by microscopy for the growth of single clones. Each well containing a single colony of cells was treated with trypsin, and suspended cells were re-plated in duplicate 96-well plates. Clones were then induced with doxycycline and screened, by immunofluorescence, for expression of dengue E protein. Several positive clones were expanded, transfected with a GFP reporter replicon, and their production of infectious reporter virus particles quantified by infection of target cells 72-168 hours later, as described herein. A number of clones generated a greater yield of RVPs compared with pooled producer cells (FIG. 14). The time after replicon transfection at which the maximum RVP yield was obtained varied for each clone.

### EXAMPLE 19

### Use of a DEN Reporter Virus Particle Carrying a Beta-Galactosidase Replicon

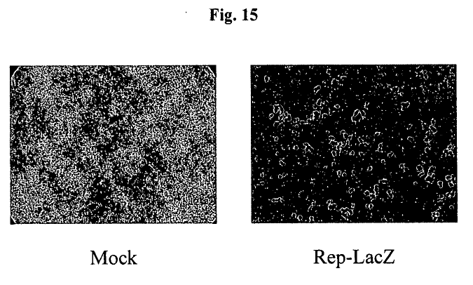

DEN RVPs can be produced using a variety of reporter proteins, including beta-galactosidase. A monocystronic, DNA-launched Dengue subgenomic beta-galactosidase replicon was constructed using plasmids described herein. Briefly, the beta-galactosidase gene was amplified from the commercially-available plasmid pCMV lacZNLS12co (Markergene Technology, Eugene, Oreg.) using the primers AAAAACGCGTATGGGCGGTAGGCGTGTACGGTGGGAGGTC (sense beta-gal) (SEQ ID NO:26) and TTTACGCGTCTTCTGGCACCACACCAGCTGGTAGTGGTAG (antisense beta-gal) (SEQ ID NO:27) and Platinum Taq High Fidelity DNA Polymerase (Invitrogen) under conditions recommended by the manufacturer. The resulting PCR product was digested using MluI and ligated to a complementary 12,644 bp fragment of the reporter replicon pDR2AH GFP, to generate pDR2AH LacZ, in which the GFP reporter has been substituted with the beta-galactosidase reporter. The resulting plasmid was transfected into 293T cells using Lipofectamine 2000 using the manufacturer's conditions. Mock- and pDR2AH LacZ-transfected cells were assayed for beta-galactosidase activity approximately 72 hours later by fixing them with 2% paraformaldehyde and incubating them with a standard X-Gal staining solution. Approximately one hour later, cells were imaged and the expression of beta-galactosidase was clearly observed (FIG. 15), demonstrating that cells can express the delivered reporter replicon. Beta-galactosidase DEN RVPs were then produced by transfection of 293trk producer cell lines with the pDR2AH LacZ plasmid. Culture media was changed 24 hours after transfection and replaced with RVP production medium (DMEM-10% FCS, 1% penicillin/streptomycin solution, 2 mM L-alanyl L-glutamine solution, 25 mM HEPES, 1 μg/ml doxycycline, pH 8.0). Supernatants were harvested at 24 hour intervals, and used to infect Raji DC-SIGNR target cells. After 72 hours, Raji DC-SIGNR cells were fixed in 2% paraformaldehyde and stained with X-Gal staining solution. Cells infected by RVPs were quantifiably identified by blue staining resulting from beta-galactosidase activity. This demonstrates that the reporter replicon can be delivered to target cells using RVPs, and can be used to quantify target cell infection.

The disclosures of each and every patent, patent application, publication, and accession number cited herein are hereby incorporated herein by reference in their entirety. The appended sequence listing is hereby incorporated herein by reference in its entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A plasmid DNA molecule comprising a nucleic acid encoding a replicon of Dengue Virus (DEN) under the control of a eukaryotic promoter.

2. The DNA molecule of claim 1, wherein said DNA molecule is free of nucleic acid encoding at least one full-length structural protein of DEN.

3. The DNA molecule of claim 1, wherein said DNA molecule comprises nucleic acid encoding at least a portion of one structural protein of DEN selected from the group consisting of C, prM, E.

4. The DNA molecule of claim 1, wherein said DNA molecule comprises nucleic acid encoding a reporter.

5. The DNA molecule of claim 4, wherein said reporter is selected from the group consisting of a GFP reporter, a Renilla luciferase reporter, and a beta-galactosidase reporter.

6. A method of producing DEN reporter virus particles (RVPs) comprising the step of contacting a cell in reporter virus particle media with a DNA molecule encoding a replicon of DEN and a reporter, wherein said cell takes up the DNA molecule, expresses said replicon of DEN and said reporter, and produces DEN RVPs.

7. The method of claim 6, wherein the said DNA molecule comprising a replicon of DEN is a plasmid.

8. The method of claim 6, wherein the reporter virus particle media is maintained at a pH of about 7.5 to about 8.5.

9. The method of claim 6, wherein the reporter virus particle media is maintained at pH of about 8.

10. The method of claim 7, wherein said contacting comprises transfection of said plasmid.

11. The method of claim 6, wherein said DNA molecule is free of nucleic acid sequences encoding at least one full-length structural protein of DEN.

12. The method of claim 6, wherein said cell stably expresses or inducibly expresses the C, prM, and E proteins of DEN.

13. The method of claim 6, wherein the DEN RVPs are harvested between 72 hours and 148 hours after contact between said DNA molecule and said cell.

14. A cell comprising structural proteins of DEN and none of the non-structural proteins of DEN.

15. The cell of claim 14 wherein said structural proteins are selected from the group consisting of C, prM, E, and combinations thereof.

16. The cell of claim 14 comprising an inducible promoter controlling the expression of said structural proteins.

17. The cell of claim 14 comprising stable integration of said structural genes and inducible promoter.

18. A method of producing DEN RVPs comprising the steps of:
a) contacting a cell in reporter virus particle media with the DNA molecule of claim 1 wherein said cell comprises (i) nucleic acids that encode DEN structural proteins; and (ii) an inducible promoter that controls the expression of DEN structural proteins;
b) inducing expression of DEN structural proteins in said cells, wherein said inducing expression of DEN structural proteins produces said RVPs.

19. The method of claim 18 wherein said cell is in reporter virus particle media that is maintained at pH of about 7.5 to about 8.5 during RVP production.

20. The method of claim 18 wherein said pH is about 8.

21. The method of claim 18, wherein the DEN RVPs are harvested between 72 hours and 148 hours after contact between said DNA molecule and said cell.

22. A composition comprising a DEN RVP and a storage buffer, wherein said storage buffer comprises an additive.

23. The composition of 22, wherein said storage buffer comprises 25 mM Hepes at about pH 8 and wherein said additive comprises about 20% fetal calf

24. The composition of 22, wherein said storage buffer comprises 25 mM Hepes at about pH 8 and wherein said additive comprises about 5% BSA.

25. The composition of 22, wherein the total additive concentration of the storage buffer is 8 ug per ml upon addition of said protein additive.

26. A method of infecting a cell comprising contacting said cell with a DEN RVP.

27. The method of claim 26, wherein said cell expresses DC-SIGNR.

28. The method of claim 26, wherein said cell expresses DC-SIGNR is a Raji-DC-SIGNR cell.

29. The method of claim 28, wherein said DEN RVP is contacted with said cell in conditions comprising about 0.5% serum.

30. A method of identifying a compound that inhibits DEN infection comprising
a) contacting a cell with a DEN RVP in the presence or absence of a test compound; and
b) determining if said DEN RVP can infect said cell in the presence and absence of said test compound
wherein if the presence of said test compound inhibits the DEN RVP infection of said cell, said test compound is said to be a compound that inhibits DEN infection.

31. A method of identifying a compound that inhibits DEN assembly comprising contacting a DEN RVP producer cell with a test compound and determining if the DEN RVPs can assemble in the presence of said test compound, wherein if assembly is prevented said test compound is said to be a compound that inhibits DEN assembly.

32. A method of identifying a compound that inhibits DEN RNA replication comprising contacting a cell containing a DEN replicon with a test compound and measuring replicon replication, wherein a decrease in replicon replication indicates that said test compound is a compound that inhibits DEN RNA replication.

33. The method of claim 32, wherein replicon replication is measured by the expression of a reporter gene.

34. The method of claim 33, wherein said reporter gene is GFP, luciferase, or beta-galactosidase.

35. A method of identifying neutralizing antibodies against DEN comprising
a) contacting a DEN RVP with a composition comprising a test antibody;
b) contacting the mixture of a) with a cell; and
c) measuring the infection of said cell in the presence of said test antibody as compared to the absence of said test antibody,
wherein a decrease in infection in the presence of said test antibody indicates that said test antibody is a neutralizing antibody against DEN virus.

36. The method of claim 35 wherein the DEN RVP comprises a nucleic acid sequence that encodes GFP, luciferase, or beta-galactosidase.

37. The method of claim 35, wherein said test antibody is a serotype-specific DEN antibody and said DEN RVP is a serotype-specific DEN RVP.

38. The method of claim 35 wherein said composition comprises patient serum.


SEQUENCE LISTING (27 sequences)

SEQ ID NO: 1 - DNA, 45 bp, Artificial Sequence
  wd1 primer sequence; synthesized product
ttttttccaaagctatggtcaatattggccattagccatattatt

SEQ ID NO: 2 - DNA, 42 bp, Artificial Sequence
  wd2 primer sequence; synthesized product
gctgcgtgaattcattcctataggaccagggttactttcaac

SEQ ID NO: 3 - DNA, 42 bp, Artificial Sequence
  wd3 primer sequence; synthesized product
gttgaaagtaaccctggtcctataggaatgaattcacgcagc

SEQ ID NO: 4 - DNA, 33 bp, Artificial Sequence
  wd4 primer sequence; synthesized product
ccacaggtaccatgctgctgccgtgattggtat

SEQ ID NO: 5 - DNA, 50 bp, Artificial Sequence
  dr1 primer sequence; synthesized product
tttttcagagctcgtttagtgaaccgagttgttagtctacgtggaccgac

SEQ ID NO: 6 - DNA, 32 bp, Artificial Sequence
  dr2 primer sequence; synthesized product
aagttacgcgtggacacgcggtttctctcgcg

SEQ ID NO: 7 - DNA, 27 bp, Artificial Sequence
  drep 1 primer sequence; synthesized product
gaagccctaggattcttaaatgaagat

SEQ ID NO: 8 - DNA, 42 bp, Artificial Sequence
  drep 2 primer sequence; synthesized product
aggctgggaccatgccggccagaacctgttgattcaacagca

SEQ ID NO: 9 - DNA, 42 bp, Artificial Sequence
  drep 3 primer sequence; synthesized sequence
tgctgttgaatcaacaggttctggccggcatggtcccagcct

SEQ ID NO: 10 - DNA, 38 bp, Artificial Sequence
  drep 4 primer sequence; synthesized product
ttatcatcgattaccacatttgtagaggttttacttgc

SEQ ID NO: 11 - DNA, 39 bp, Artificial Sequence
  ren 5′ primer sequence; synthesized product
aaaaaaacgcgtatggcttcgaaagtttatgatccagaa

SEQ ID NO: 12 - DNA, 40 bp, Artificial Sequence
  ren 3′ primer sequence; synthesized product
aaaaaaggcgcgccgtgatagatctttgttcatttttgag

SEQ ID NO: 13 - DNA, 40 bp, Artificial Sequence
  d27 primer sequence; synthesized product
aaaaaaacgcgtatggtgagcaagggcgaggagctgttca

SEQ ID NO: 14 - DNA, 37 bp, Artificial Sequence
  d28 primer sequence; synthesized product
tttttacgcgtgtcctgctcctcggccacgaagtgca

SEQ ID NO: 15 - DNA, 34 bp, Artificial Product
  d29 primer sequence; synthesized product
ttggccttgtacagctcgtccatgccgagagtga

SEQ ID NO: 16 - DNA, 36 bp, Artificial Sequence
  d30 primer sequence; synthesized product
acaaggccaagttgaccagtgccgttccggtgctca

SEQ ID NO: 17 - DNA, 40 bp, Artificial Sequence
  d31 primer sequence; synthesized product
aaaaaaacgcgtatgacttcgaaagtttatgatccagaac

SEQ ID NO: 18 - DNA, 37 bp, Artificial Sequence
  d32 primer sequence; synthesized product
ttggcttgttcatttttgagaactcgctcaacgaacg

SEQ ID NO: 19 - DNA, 40 bp, Artificial Sequence
  d33 primer sequence; synthesized product
aacaagccaagttgaccagtgccgttccggtgctcaccgc

SEQ ID NO: 20 - DNA, 29 bp, Artificial Sequence
  d1 primer sequence; synthesized product
caccatgaataaccaacggaaaaaggcga

SEQ ID NO: 21 - DNA, 36 bp, Artificial Sequence
  d2 primer sequence; synthesized product
tttcactattaggcctgcaccatgactcccaaatac

SEQ ID NO: 22 - DNA, 31 bp, Artificial Sequence
  d3 primer sequence; synthesized product
caccatgaacaaccaacggaaaaagacgggt

SEQ ID NO: 23 - DNA, 36 bp, Artificial Sequence
  d4 primer sequence; synthesized product
tttcactattacgcctgaaccatgactcctaggtac

SEQ ID NO: 24 - DNA, 29 bp, Artificial Sequence
  d5 primer sequence; synthesized product
caccatgtctgcaggcatgatcattatgc

SEQ ID NO: 25 - DNA, 30 bp, Artificial Sequence
  d6 primer sequence; synthesized product
caccatgtctgtgaccatgctcctcatgct

SEQ ID NO: 26 - DNA, 40 bp, Artificial Sequence
  beta-gal sense primer sequence; synthesized product
aaaaacgcgtatgggcggtaggcgtgtacggtgggaggtc

SEQ ID NO: 27 - DNA, 40 bp, Artificial Sequence
  beta-gal antisense primer sequence; synthesized product
tttacgcgtcttctggcaccacaccagctggtagtggtag